(19) United States
(12) Patent Application Publication
(10) Pub. No.: US 2014/0314788 A1
(43) Pub. Date: Oct. 23, 2014

(54) MODULATION OF HISTONE DEACETYLASES FOR THE TREATMENT OF METABOLIC DISEASE, METHODS AND COMPOSITIONS RELATED THERETO

(71) Applicants: Reuben J. Shaw, San Diego, CA (US); Maria Mihaylova, San Diego, CA (US); Marc R. Montminy, San Diego, CA (US); Kim Ravnskjaer, La jolla, CA (US); Biao Wang, Carlsbad, CA (US)

(72) Inventors: Reuben J. Shaw, San Diego, CA (US); Maria Mihaylova, San Diego, CA (US); Marc R. Montminy, San Diego, CA (US); Kim Ravnskjaer, La jolla, CA (US); Biao Wang, Carlsbad, CA (US)

(73) Assignee: Salk Institute for Biological Studies, La Jolla, CA (US)

(21) Appl. No.: 13/640,261

(22) PCT Filed: Apr. 11, 2011
(86) PCT No.: PCT/US11/31991

§ 371 Date: Jun. 12, 2013

Related U.S. Application Data
(60) Provisional application No. 61/322,625, filed on Apr. 9, 2010.

(51) Int. Cl. G01N 33/50; C07K 16/40; C12N 15/113

(52) U.S. Cl. CPC G01N 33/5023; C12N 15/1137; C07K 16/40
USPC 424/158.1; 514/44 A; 435/18

(57) ABSTRACT

The invention relates to methods and compositions for the modulation of glucose homeostasis and/or the treatment of metabolic diseases. In some embodiments, the invention relates to methods and compositions for the modulation of histone deacetylases. such as Class IIa histone deacetylases.

### CROSS-REFERENCE TO RELATED APPLICATION

This application claims the benefit of priority of U.S. Provisional Application Ser. No. 61/322,625, filed Apr. 9, 2010. The foregoing application is incorporated herein by reference in its entirety.

### FIELD OF THE INVENTION

The invention relates generally to methods and compositions for the modulation of glucose homeostasis and/or the treatment of metabolic diseases. More particularly, the invention relates to methods and compositions for the modulation of histone deacetylases, such as Class IIa histone deacetylases.

### BACKGROUND OF THE INVENTION

How multicellular organisms store and utilize nutrients in response to changing environmental conditions is under the control of hormones, as well as cell-autonomous nutrient and energy sensors. Glucose homeostasis in mammals is primarily maintained through a tight regulation of glucose uptake in peripheral tissues in the fed state and production of glucose in liver during fasting. After a meal, insulin signals the liver to attenuate glucose production and the muscle and adipose to increase glucose uptake. Conversely, in the fasted state, glucagon signals the liver to upregulate gluconeogenesis, to ensure constant blood glucose levels. Dysregulation of these processes contributes to metabolic disorders such as Type 2 diabetes (Biddinger and Kahn, 2006).

Gluconeogenesis is largely regulated at the transcriptional level of rate-limiting enzymes including glucose-6-phophatase (G6pc; G6Pase) and phosphoenolpyruvate carboxykinase (Pck1; PEPCK) via hormonal modulation of transcription factors and coactivators including CREB, FOXO, HNF4α, GR, PGC1α, and C/EBPs (Viollet et al., 2009). Two major signaling pathways suppressing gluconeogenic transcription are the insulin signaling pathway and the LKB1/AMPK pathway. Insulin control of gluconeogenesis is largely mediated through the serine/threonine kinase Akt, which phosphorylates and inactivates PGC1α and the FOXO family of transcription factors, mainly Foxo1 and Foxo3 in mammalian liver (Matsumoto et al., 2007; Haeusler et al., 2010). Akt-dependent phosphorylation inactivates FOXO through 14-3-3 binding and subsequent cytoplasmic sequestration. In addition, FOXO is inhibited through acetylation on up to 6 lysines, which reduces its DNA-binding ability and alters its subcellular localization. The Akt sites and acetylation sites are well conserved in metazoans and across Foxo family members (Calnan and Brunet, 2008).

The LKB1/AMPK pathway is also a significant endogenous inhibitor of gluconeogenesis (Shaw et al., 2005; Viollet et al., 2009; Canto and Auwerx, 2010). LKB1 is a master upstream kinase that directly phosphorylates the activation loop of 14 kinases related to the AMP-activated protein kinase (AMPK). In liver, AMPK activity is modulated by adipokines such as adiponectin, but not thought to be regulated during physiological fasting by blood glucose levels as they rarely fall low enough to trigger ATP depletion (Kahn et al., 2005). However, a number of pharmacological agents that trigger mild ATP depletion by disrupting mitochondrial function can activate AMPK, including the biguanide compounds phenformin and metformin, which is the most widely used type 2 diabetes therapeutic worldwide. In addition to AMPK, at least two other related LKB1-dependent kinases can also suppress gluconeogenesis: Salt-Inducible Kinase 1 (SIK1) and SIK2 (Koo et al., 2005). These LKB1-dependent kinases can all phosphorylate common downstream substrates to inhibit gluconeogenesis, of which the CRTC2 coactivator is one example, though it is likely that additional targets exist (Shackelford and Shaw, 2009).

In addition to protein phosphorylation, acetylation of histones and transcription factors is also modulated during the fasting and feeding response in liver (Guarente, 2006). Three families of deacetylases counteract the actions of the acetyltransferases (HATs). Class I HDACs (HDAC1, 2, 3, and 8) are thought to be classical histone deacetylases, though recently these have been found to be associated with active transcriptional regions (Wang et al., 2009) and non-histone targets have been reported (Gregoire et al., 2007; Canettieri et al., 2010). Class IIa HDACs (HDAC4, 5, 7 and 9) are thought to be catalytically inactive due to critical amino acid substitutions in their active site (Haberland et al., 2009), and are proposed to act as scaffolds for catalytically active HDAC3-containing complexes in several settings (Wen et al., 2000; Fischle et al., 2002). Similar to FOXO, the localization of Class IIa HDACs to the nucleus is inhibited through phosphorylation on specific conserved residues (Ser 259 and Ser498 in human HDAC5), and subsequent 14-3-3 binding resulting in cytoplasmic sequestration (reviewed in Haberland et al., 2009). Based on their homology to Sir2 in budding yeast, the Class III family of HDACs are also known as Sirtuins, and several mammalian Sirtuins are activated by NAD+ and thus serve as energy sensors (Houtkooper et al., 2010; Haigis and Sinclair, 2010).

The Class IIa family of histone deacetyltransferases (HDACs) are highly conserved substrates of AMPK family kinases (Gwinn, D M et al.). Multiple candidate phosphorylation sites in the Class IIa HDACs are conserved back through Drosophila and C. elegans and represent the well-established phosphorylation sites governing their subcellular localization via 14-3-3 binding (McKinsey, T A et al.; Grozinger, C M et al.; Vega, R B et al.; Bordeaux, R et al.).

Class IIa HDACs are signal-dependent modulators of transcription with established roles in muscle differentiation and neuronal survival (Haberland, M. et al.; Martin, M. et al.). They have been thought to be expressed the highest in brain and cardiac and skeletal muscle (Chang, S et al.).

### SUMMARY OF THE INVENTION

The present invention provides a method of treating a metabolic disorder, comprising administering to a subject in need thereof an inhibitor of a Class IIa histone deacetylase (HDAC). In certain embodiments, the Class IIa HDAC is selected from the group consisting of: HDAC 4, 5, 7, and 9. In some embodiments, the Class IIa HDAC inhibitor inhibits the activity of two or more Class IIa HDACs selected from the group consisting of: HDAC 4, 5, 7, and 9. In further embodiments, the Class IIa HDAC inhibitor inhibits the activity of HDAC 4, 5, 7, and 9. In some embodiments, the metabolic disorder is selected from the group consisting of diabetes, insulin resistance, and obesity. In certain embodiments, the diabetes is selected from the group consisting of: type 1 diabetes, type 2 diabetes, and gestational diabetes.

In some embodiments, the invention relates to a method of a treating a metabolic disorder, comprising administering to a subject in need thereof, a Class IIa HDAC inhibitor, wherein the Class IIa inhibitor lowers blood glucose levels in the subject.

In some embodiments, the invention relates to a method of treating a metabolic disorder, comprising administering to a subject in need thereof, a Class IIa HDAC inhibitor, wherein the Class IIa HDAC inhibitor inhibits an activity of a Class IIa HDAC. In some embodiments, the Class IIa HDAC inhibitor inhibits expression of a Class IIa HDAC. In certain embodiments, the Class IIa HDAC inhibitor inhibits nuclear localization of a Class IIa HDAC. In other embodiments, the Class IIa HDAC inhibitor inhibits dephosphorylation of a Class IIa HDAC. In yet other embodiments, the Class IIa HDAC inhibitor inhibits Class IIa HDAC mediated deactylation of a protein involved in glucose homeostasis, such as a FOXO transcription factor.

Examples of Class IIa inhibitors include MC1568 and pharmaceutically acceptable salts thereof. In certain embodiments, the invention provides a method of treating a metabolic disorder, comprising administering to a subject in need thereof C1568 or a pharmaceutically acceptable salt thereof. In certain embodiments, administration of MC1568 lowers blood glucose levels in the subject. In some embodiments, the metabolic disorder is diabetes. In further embodiments, the diabetes is selected from the group consisting of type 1 diabetes, type 2 diabetes, and gestational diabetes.

In some embodiments, the invention provides a method of screening for a compound for treating a metabolic disorder, comprising expressing a Class IIa HDAC in an isolated hepatocyte or in the liver of a non-human animal in the absence and presence of a test compound, and evaluating the activity of the Class IIa HDAC in the absence and presence of the test compound. In certain embodiments, the activity of the Class IIa HDAC is selected from the group consisting of: expression, localization, phosphorylation state, and FOXO transcription factor deacetylation.

In yet other embodiments, the invention provides a method of treating a muscle wasting disease, comprising administering to a subject in need thereof an inhibitor of a Class IIa histone deacetylase (HDAC). In some embodiments, the Class IIa HDAC is selected from the group consisting of HDAC 4, 5, 7, and 9. In certain embodiments, the Class IIa HDAC inhibitor inhibits the activity of at least one Class IIa HDAC selected from the group consisting of HDAC 4, 5, 7, and 9. In other embodiments, the Class IIa HDAC inhibitor inhibits the activity of HDAC 4, 5, 7, and 9. In certain embodiments, the Class IIa HDAC inhibitor inhibits the interaction of one or more Class IIa HDACs with HDAC 3. In further embodiments, the Class IIa HDAC inhibitor inhibits the interaction of HDAC 4 and/or 5 with HDAC 3. In certain embodiments, the muscle wasting disease is selected from the group consisting of: cachexia, muscle wasting, age-related sarcopenia, and muscular dystrophy.

### BRIEF DESCRIPTION OF THE DRAWINGS

FIG. 1 shows that Class IIa HDACs are regulated by LKB1-dependent kinases and metformin treatment in liver

(A) Clustal alignment of Class IIa HDACs showing sequence conservation on established phosphorylation sites matching the optimal AMPK motif.

(B) Primary mouse hepatocytes or mouse livers lysates infected with adenoviruses bearing indicated shRNAs and immunoblotted with indicated antibodies (full description in supplemental Supporting text).

(C) Lysates of HepG2 or Huh7 cells transfected with indicated siRNA pools and treated with either 2 mM Phenformin or vehicle for 1 hr and subjected to immunoblotting.

(D) Immunoblot of lysates from murine livers from LKB1+/+ or LKB1lox/lox mice deleted for hepatic LKB1 and treated with either 250 mg/kg metformin, or saline alone for 1 h.

FIG. 2 shows that glucagon induces dephosphorylation and nuclear translocation of Class IIa HDACs in hepatocytes

(A) Liver lysates from C57B1/6J mice either fasted for 18 h and/or then refed for 4 h (left panel). Mice were either fasted for 6 h or fed ad libitum (right panel).

(B) Primary mouse hepatocytes treated with 100 nM glucagon or vehicle for indicated times, lysed and immunoblotted with indicated antibodies.

(C) Primary hepatocytes were treated with either 10 uM Forskolin, 100 nM Glucagon or 100 nM Insulin for 1 h. Cells were lysed and blotted for indicated proteins. Results are representative of 3 independent experiments for each panel.

(D) Primary mouse hepatocytes infected with adenovirus expressing GFP-HDAC5 WT either treated with 100 nM glucagon or vehicle (media) for indicated times and analyzed by confocal microscopy.

FIG. 3 shows that Class IIa HDACs are required for the induction of gluconeogenic genes and associate with the G6Pase locus following glucagon

(A) Microarray data analysis on genes induced by forskolin in primary mouse hepatocytes and whose expression is altered in due to depletion of HDAC4 & 5 (HDAC) but not scrambled (scram) control shRNA. Cells were treated with 10 uM forskolin or vehicle (DMSO) for 2 or 4 h as indicated. Duplicate samples are shown for each condition. Gene expression shown relative to scrambled shRNA cells treated with vehicle for 2 h. FOXO regulated targets (Dong et al., 2006; Dansen et al., 2004; Renault et al., 2009; Paik et al., 2009) (#) or CREB regulated targets (Zhang et al., 2005) (*) as indicated. Rate-limiting gluconeogenic enzymes highlighted in red. Representative 15 of the top 50 HDAC4/5-regulated genes shown.

(B) qRT-PCR from primary hepatocytes of FOXO target genes whose FSK-induced expression is attenuated following depletion with HDAC4/5/7 shRNAs. Expression relative to cyclophilin. (n=9) *p<0.01

(C) Ad-G6Pase-luc activity (top panel) or CRE-luc activity (bottom panel) in primary hepatocytes expressing indicated adenoviruses and treated with vehicle or 10 uM Forskolin for 4 h as indicated. Representative of 4 independent experiments. (n=6) *p<0.007

(D) G6Pase-luc activity in 18 h fasted mice expressing Ad-scrambled or Ad-HDAC4/5/7 shRNAs. Results representative of 3 independent experiments and quantified using the Living Image 3.2 program. (n=6) *p<0.002

(E) Endogenous HDAC4 or HDAC5 chromatin immunoprecipitation (ChIP) with primers against indicated regions of the murine G6Pase promoter at the times indicated following 100 nM glucagon treatment. (n=4) *p<0.05

Data are shown as mean+/−s.e.m. using Student's t-test.

FIG. 4 shows that Class IIa HDACs control FOXO acetylation

(A) HEK293T cells transfected with MYC-Foxo1 and GFP-HDAC5 as indicated, treated with 10 uM forskolin or vehicle for 1 h and immunoprecipitated with anti-myc tag antibody.

(B) Primary hepatocytes treated for 1 h with vehicle or 10 uM Forskolin and endogenous Foxo1 and HDAC4 were detected by immunocytochemistry.

(C) Immunoblot showing amounts of acetylated FOXO (Ac-Lys259/262/271) from primary hepatocytes transduced with adenoviruses expressing Foxo3 and indicated shRNAs. Total cell lysates were blotted with indicated antibodies.

(D) Primary hepatocytes were transduced with adenoviruses expressing Foxo3 or GFP-FOXO1 and indicated shRNA-expressing adenoviruses. Foxo immunoprecipitates were immunoblotted with indicated antibodies.

(E) Lysates from mouse livers knockdown with either scrambled or Class IIa HDAC4/5/7 shRNAs were immunoprecipitated for endogenous Foxo1 protein and immunoblotted with indicated antibodies.

(F) Primary hepatocytes knocked down for the Class IIa HDACs or control scramble shRNAs and total cell lysates were immunoblotted with indicated antibodies.

FIG. 5 shows that Class I HDAC3 is recruited by Class IIa HDACs to deacetylate Foxo

(A) ChIP analysis on primary hepatoyctes transduced with control scramble or HDAC4/5/7 shRNAs and assessed for HDAC3 association on Foxo binding sites within G6Pase or PCK1 or the housekeeping TFIIB promoter following 1 h treatment with 100 nM Glucagon. (n=4) *p<0.05

(B) HEK293T cells transfected with a FLAG-HDAC3 and GFP-HDAC5 as indicated and treated with forskolin or vehicle for 1 h and then immunoprecipitated with anti-FLAG tag antibodies. Immunoprecipitates and input cell lysates were blotted with indicated antibodies.

(C) In vitro deacetylation assays were performed on recombinant GST-FOXO1, pre-acetylated in vitro with a recombinant fragment of p300. GST-FOXO1 acetylation is detected using the Foxo1 K242/245 acetylation specific antibody. Recombinant HDAC3 or HDAC3 complexed with Ncor was used at varying concentrations. Recombinant SIRT1 used as positive control.

(D) ChIP analysis on primary hepatoyctes transduced with control scramble or HDAC4/5/7 shRNAs and assessed for Foxo1 on G6Pase or PCK1 promoters following 1 h treatment with 100 nM Glucacon. (n=4) *p<0.05

Data are shown as mean+/−s.e.m. using Student's t-test.

FIG. 6 shows that Class IIa HDACs are required for glucose homeostasis

(A) C57B1/6J mice infected with indicated shRNAs in liver and fasted for 18 h and/or then refed for 4 h. Livers were processed for histology and stained with hematoxilin and eosin (H&E) or periodic-shiff's stain (PAS) to detect glycogen. Images were taken at 40×.

(B) qRT-PCR for G6Pase expression from livers of ad lib fed C57B1/6J mice expressing GFP or GFP-HDAC5-AA or HDAC4/5 shRNAs in liver. (n=9) *p<0.01, **p<0.001, ***p<0.0001

(C) Albumin-creERT2 LKB1+/+ or LKB1lox/lox mice were tamoxifen-treated and subsequently infected with scrambled or HDAC4/5/7 (HDAC) shRNAs. 5 days later, mice were fasted for 18 h, and blood glucose was measured. Average blood glucose value shown in red. (n=5) **p<0.001 ***p<0.0001

(D) qRT-PCR for FOXO target genes (Igfbp1, Agxt2l1, Mmd2) or Hdac4 (control) from livers of indicated mice from C. (n=9) *p<0.01, **p<0.001, ***p<0.0001. ,

(E) Liver lysates from mice in C were immunoblotted with indicated antibodies. Asterisk indicates a non-specific band recognized by the HDAC5 antibody.

Data are shown as mean+/−s.e.m. using Student's t-test.

FIG. 7 shows that suppression of Class IIa HDACs lowers blood glucose in mouse models of metabolic disease

(A) Glucose tolerance test was performed on db/db mice infected with either scramble (scramb) shRNA or HDAC4/5/7 (HDAC) shRNAs. (n=5) *p<0.02

(B) Db/db mice knocked down with (scramb) shRNA or HDAC4/5/7 (HDAC) shRNAs in liver. 5 days later, mice were fasted for 18 h and blood glucose was measured. Average blood glucose value shown in red. (n=5) *p<0.02

(C) Pyruvate tolerance test performed on db/db mice injected with either scramble (scramb) shRNA or HDAC4/5/7 (HDAC) shRNAs. (n=5) *p<0.04

(D) 7 month old B6 mice on a high fat diet (HFD) were treated as in A. (n=5) *p<0.03

(E) Glucose tolerance test was performed on 7 month old B6 mice on HFD as in C. (n=5) *p<0.02

(F) Model for Glucagon dependent regulation of Class IIa HDACs and FOXO. Under fasting conditions, glucagon induces dephosphorylation and nuclear translocation of Class IIa HDACs. Once nuclear, they associate with the G6Pase and PEPCK promoters and bind to HDAC3-Ncor/SMRT and FOXO1/3, resulting in HDAC3-mediated deacetylation and activation of FOXO. Under fed conditions insulin-dependent activation of the LKB1-dependent kinases SIK1/2 stimulates phosphorylation and cytoplasmic shuttling of Class IIa HDACs. Similarly, following metformin treatment, the LKB1-dependent AMPK activation induces Class IIa HDAC phosphorylation and 14-3-3 binding. In response to glucagon, PKA is activated and directly phosphorylates and inactivates AMPK, SIK1, and SIK2 hence resulting in loss of HDAC phosphorylation.

Data are shown as mean+/−s.e.m. using Student's t-test.

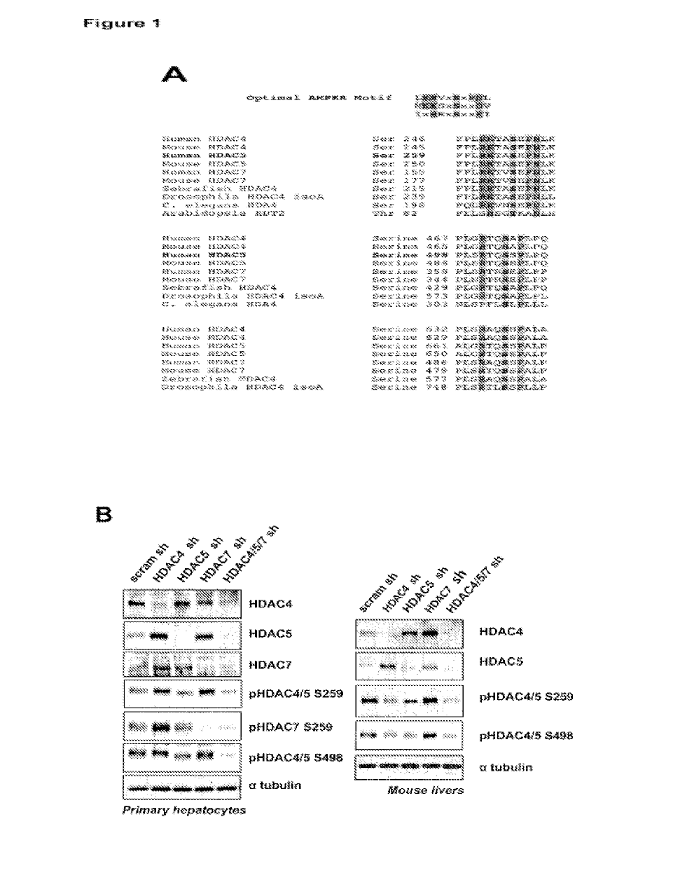

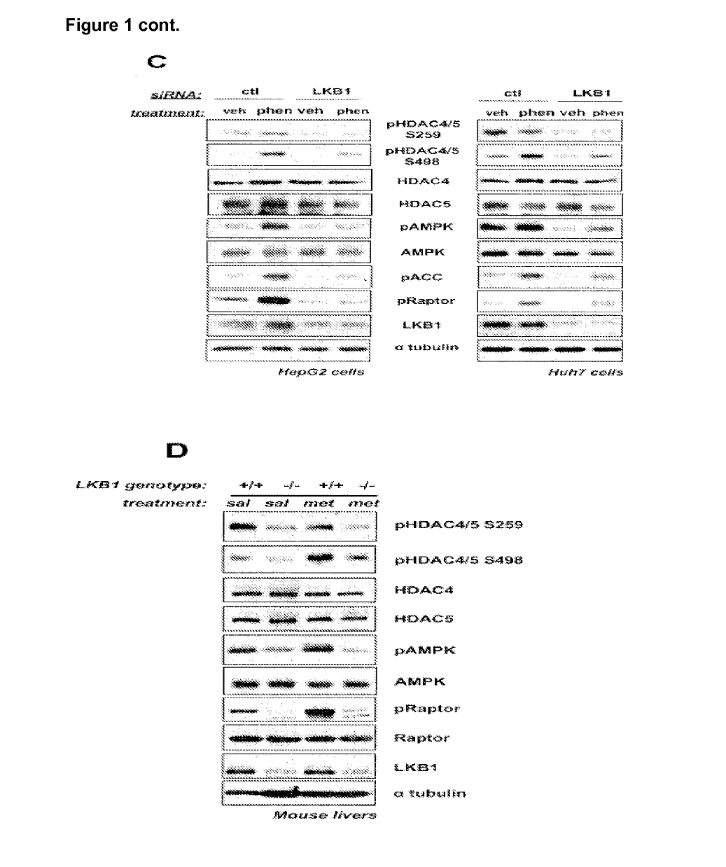

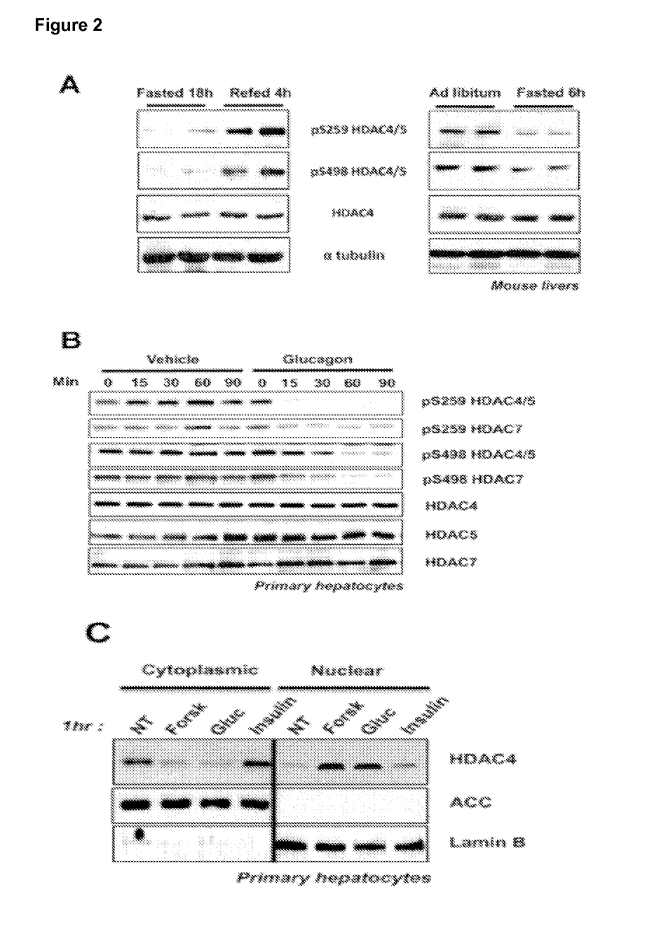

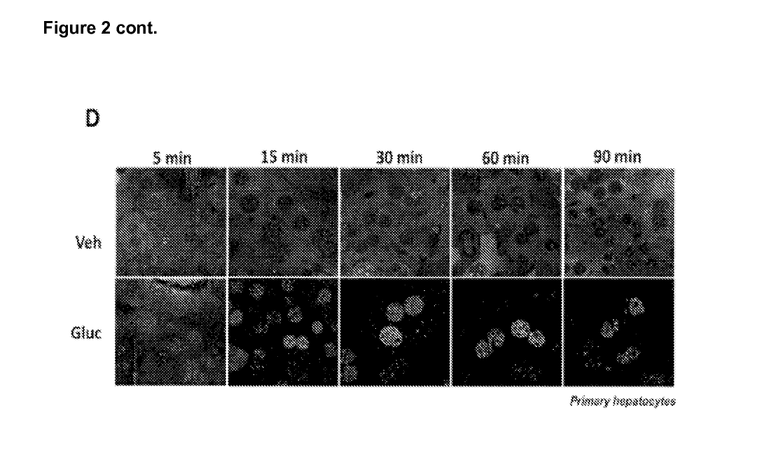

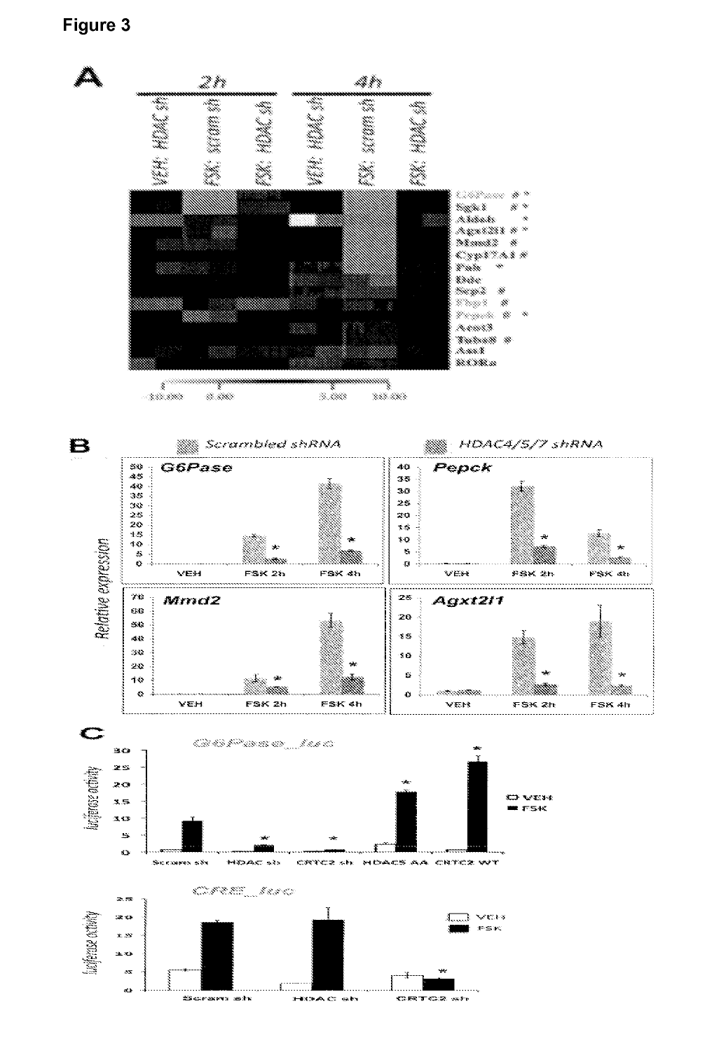

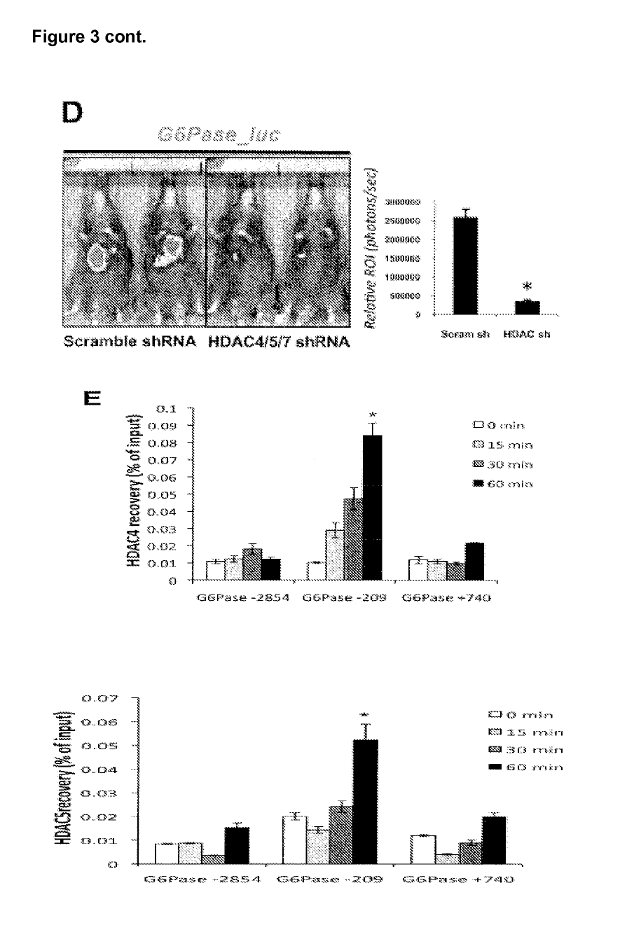

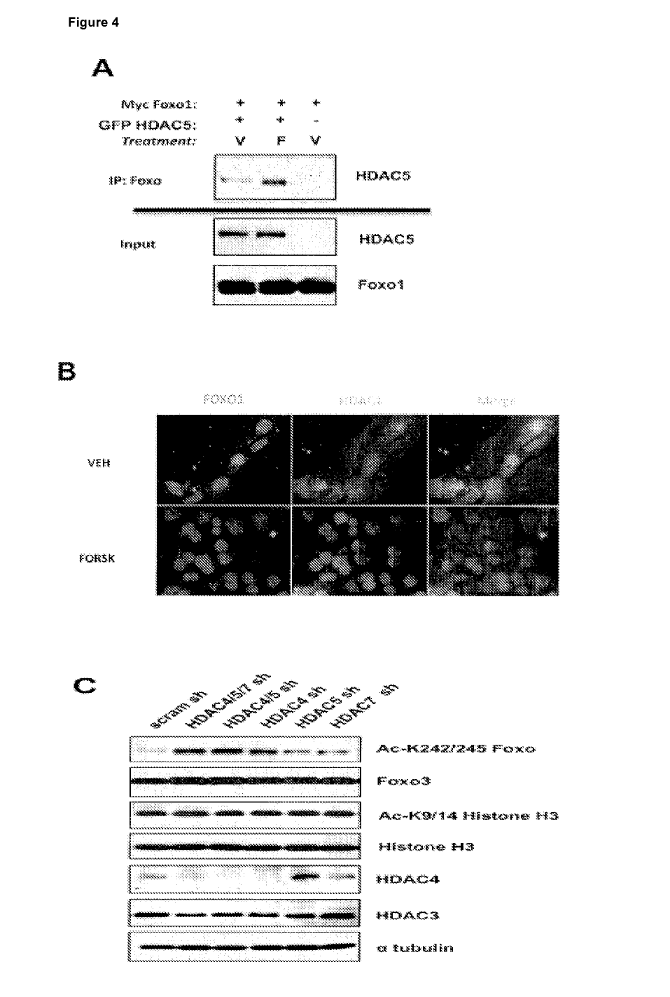

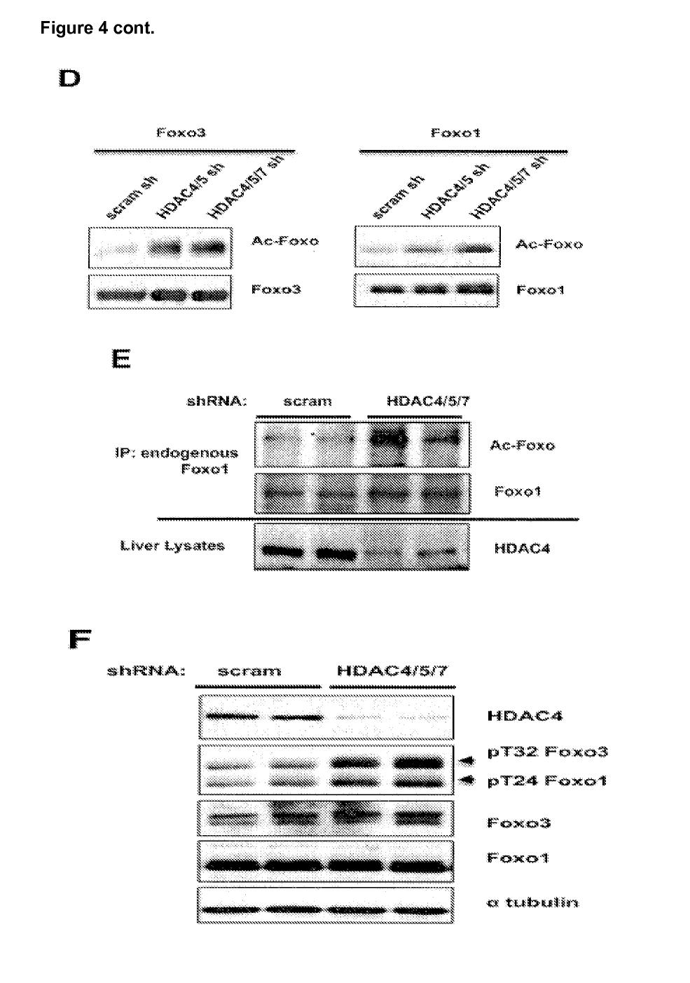

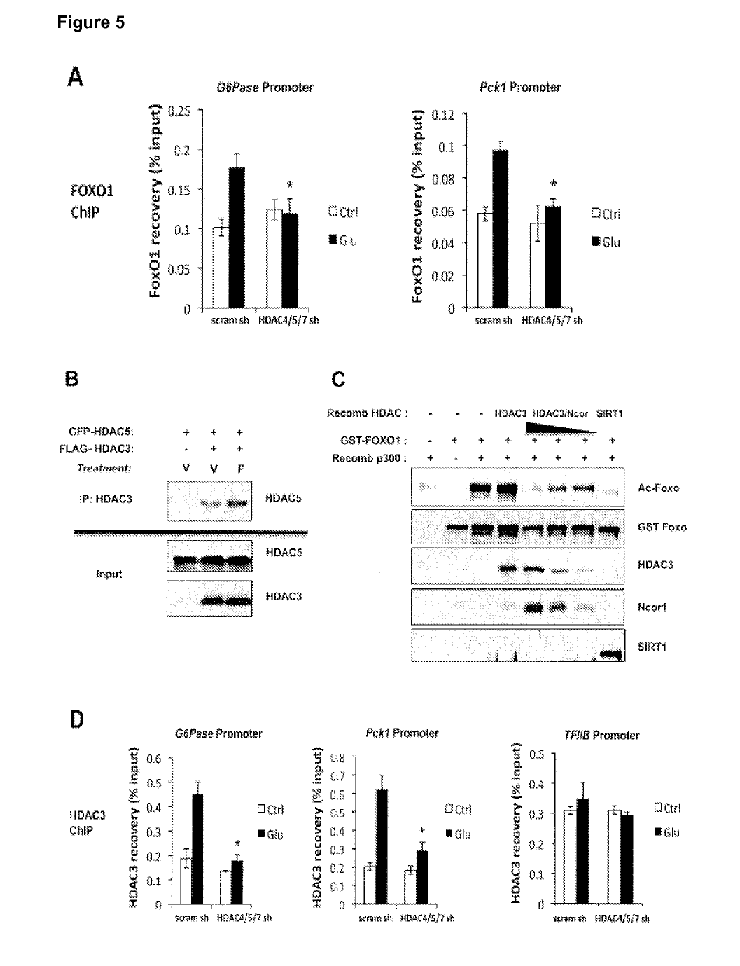

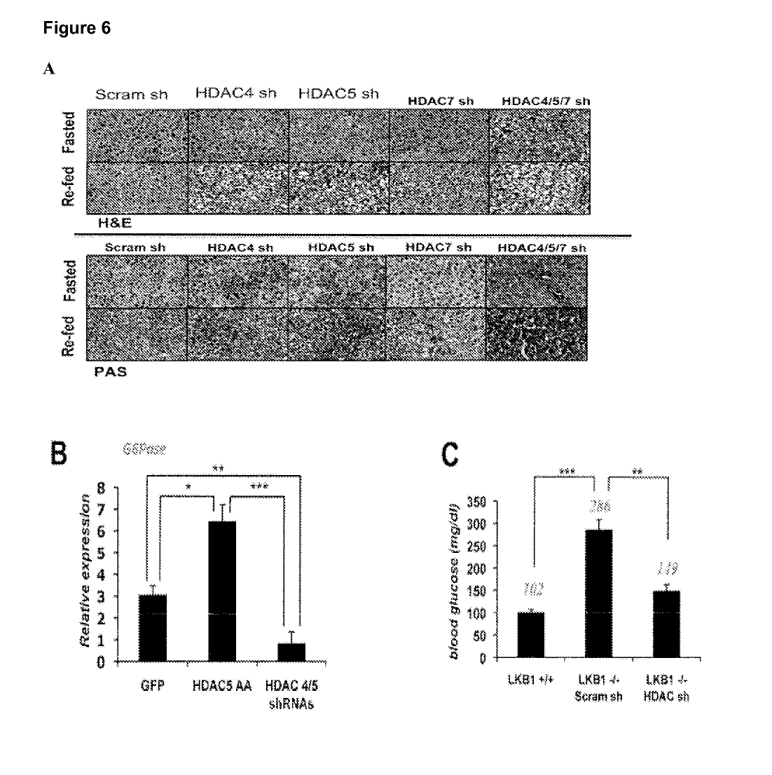

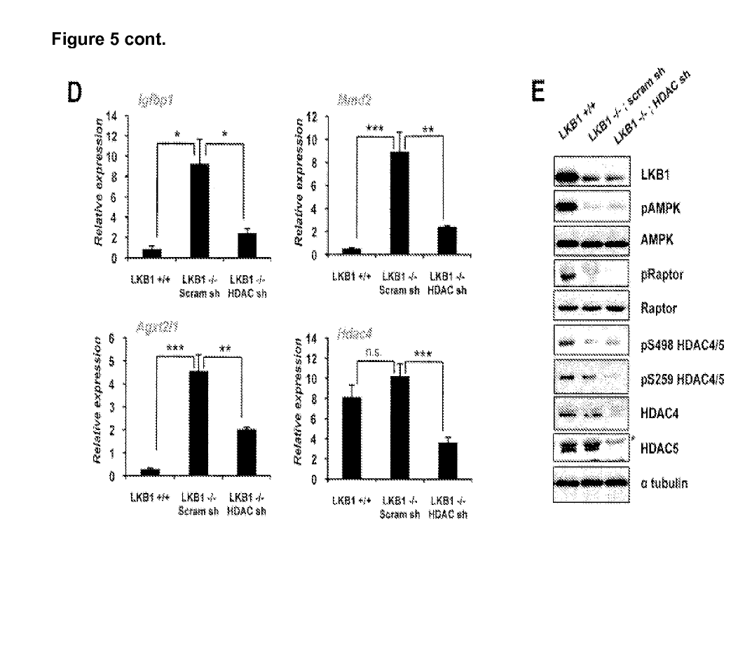

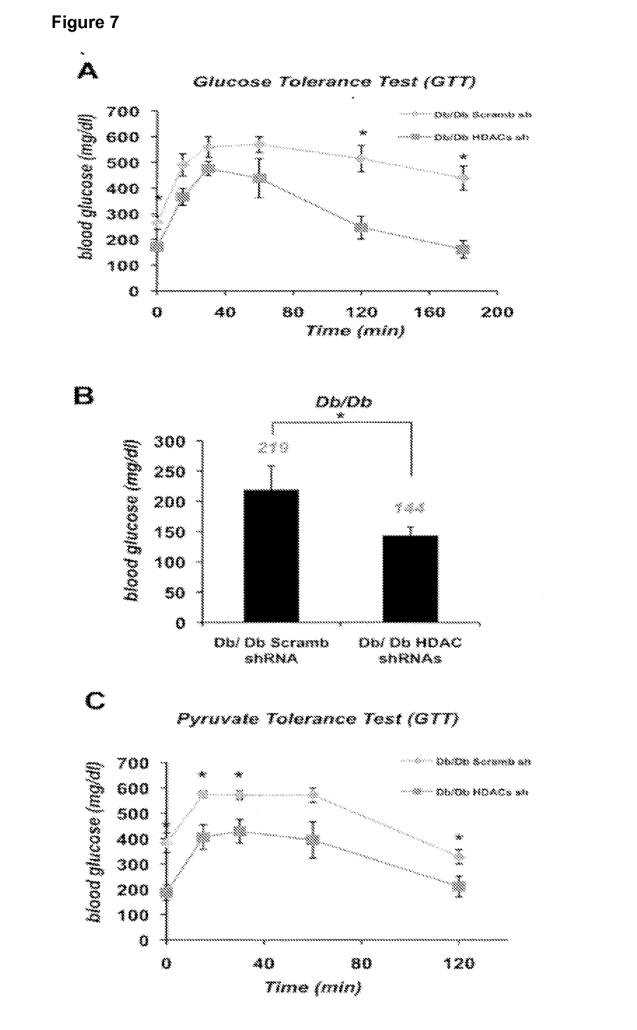

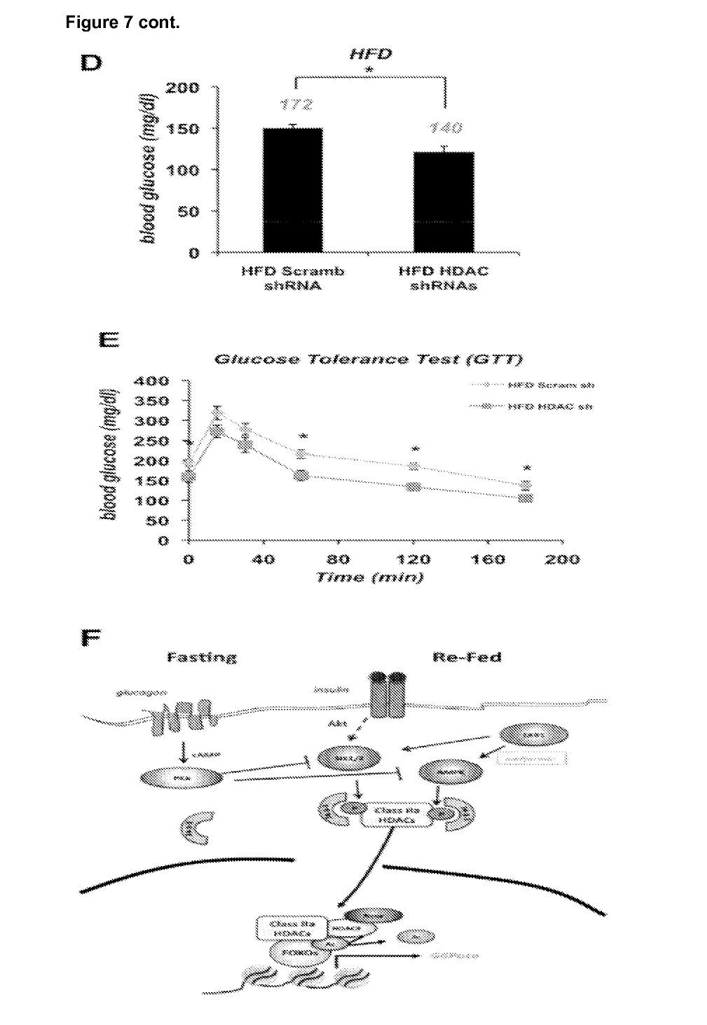

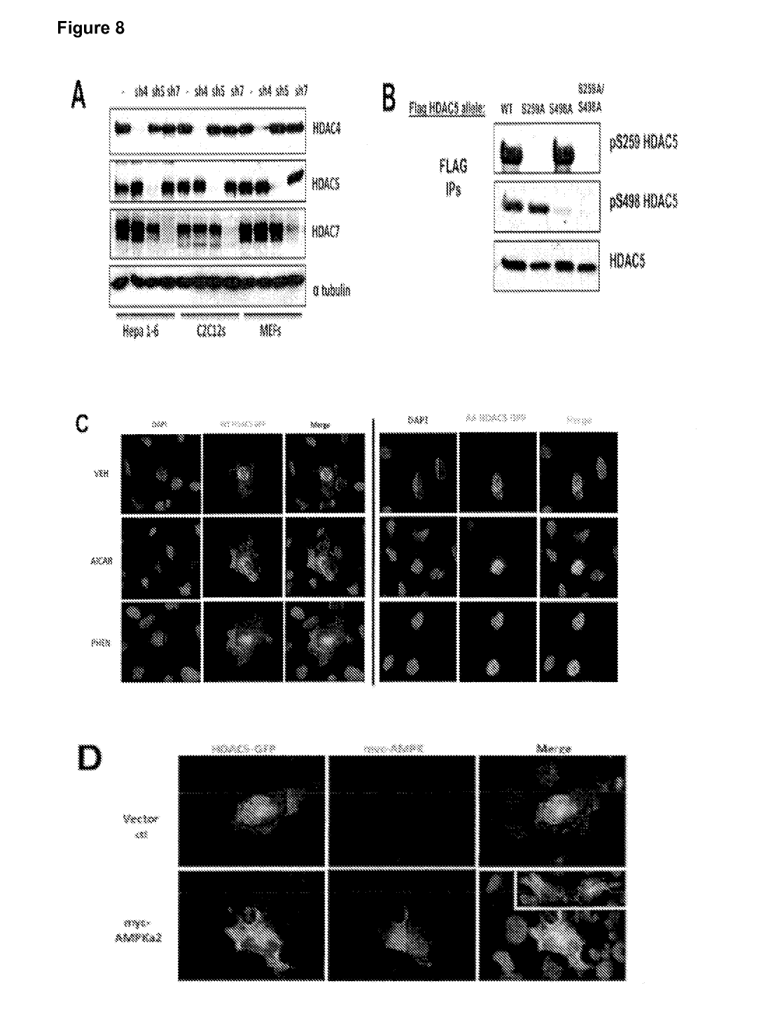

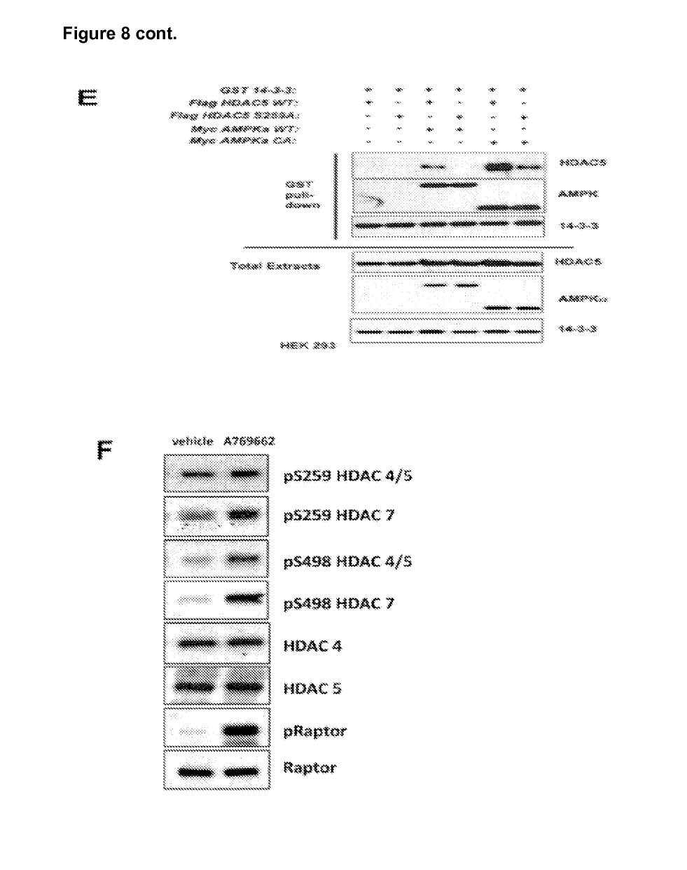

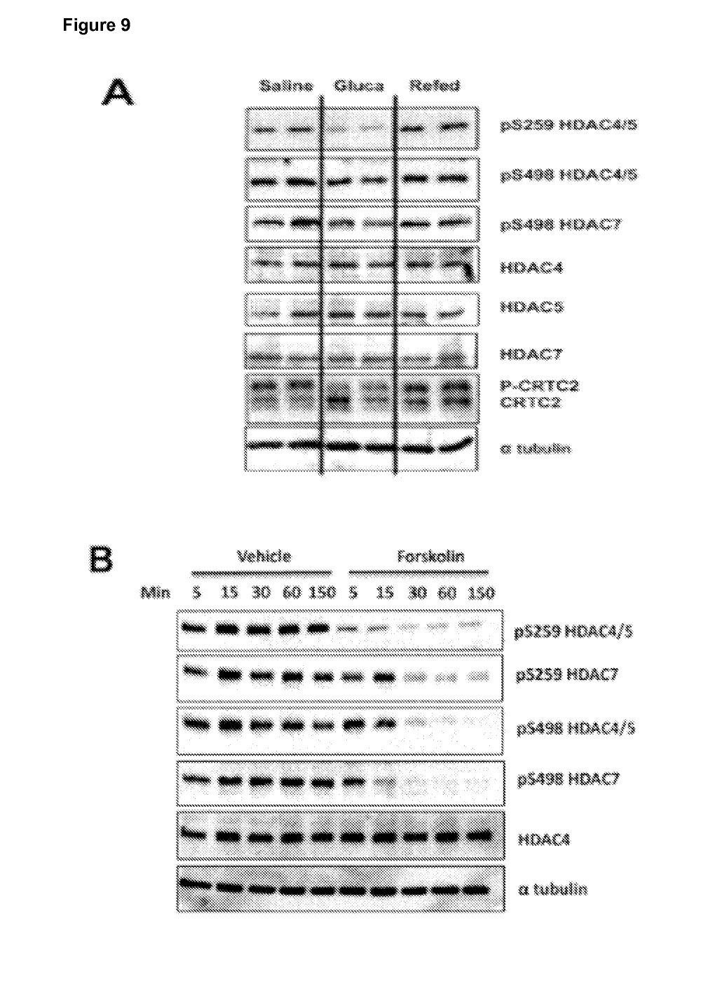

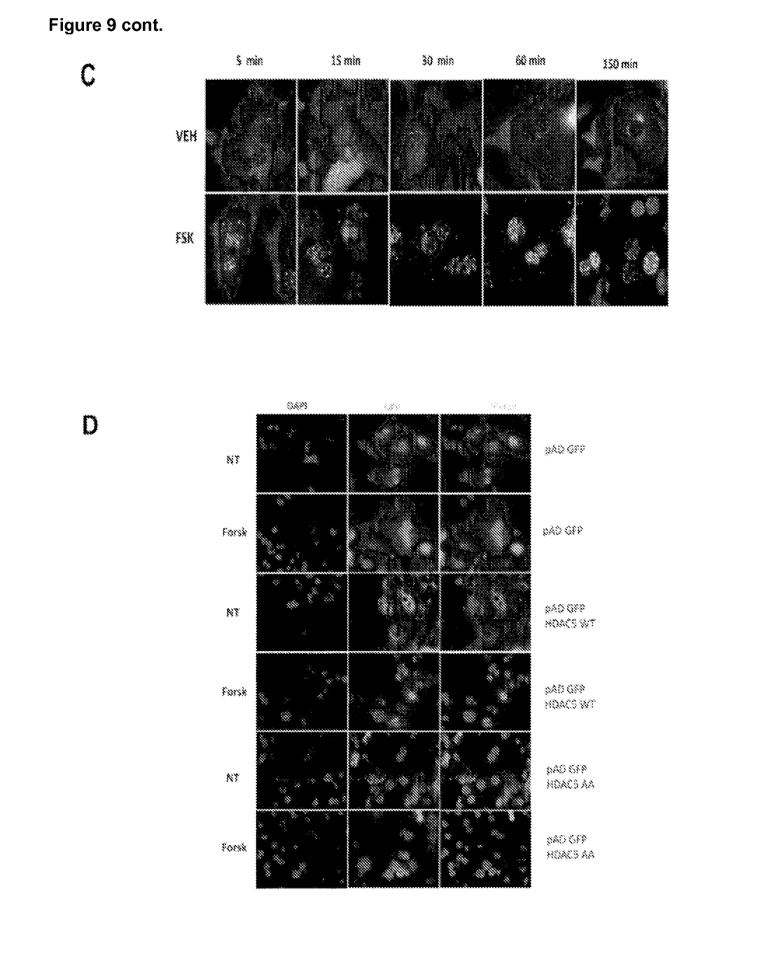

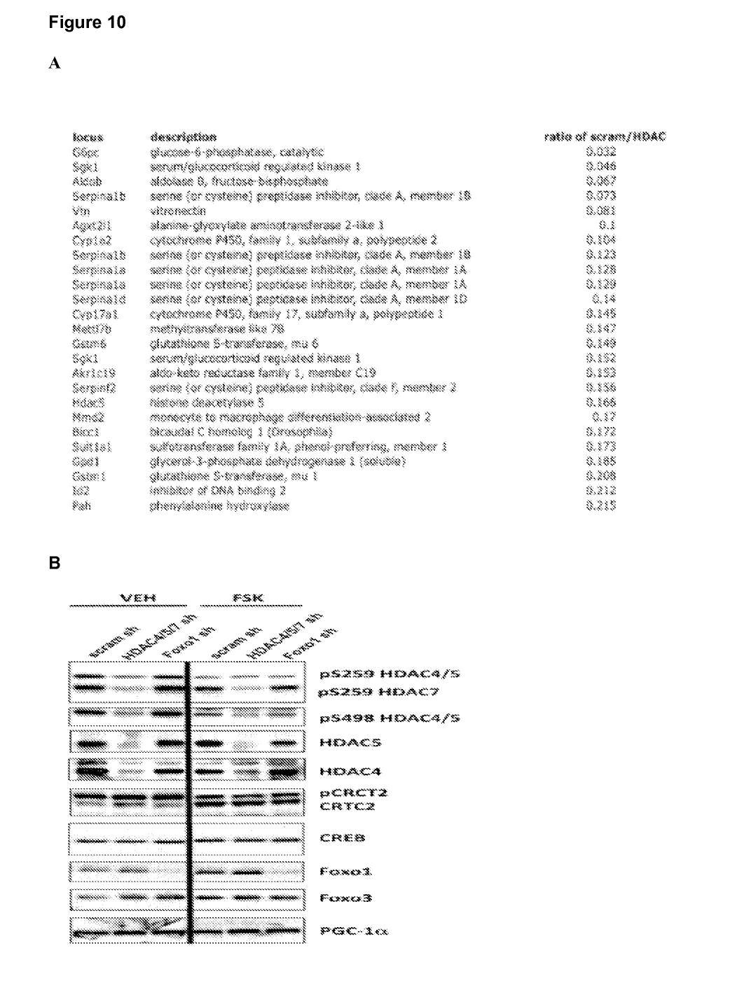

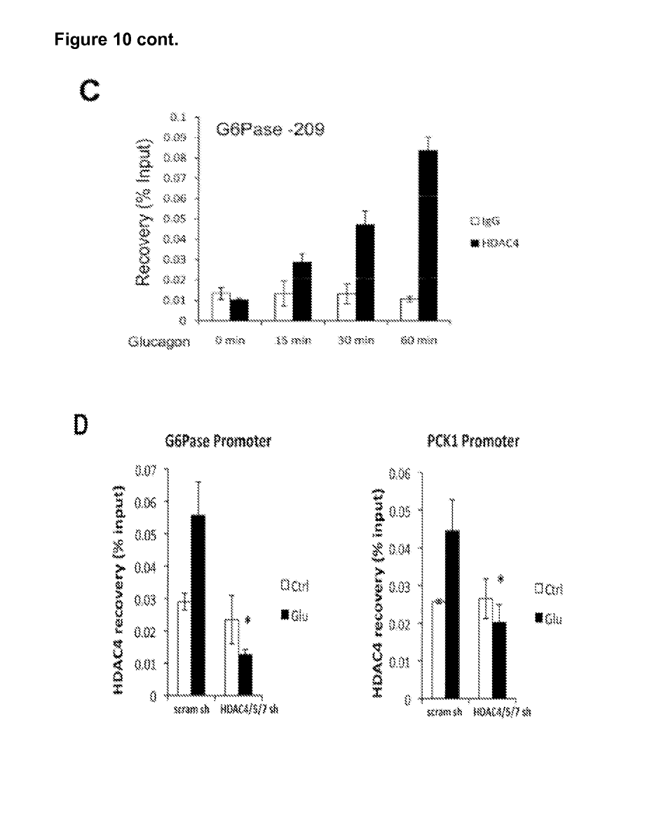

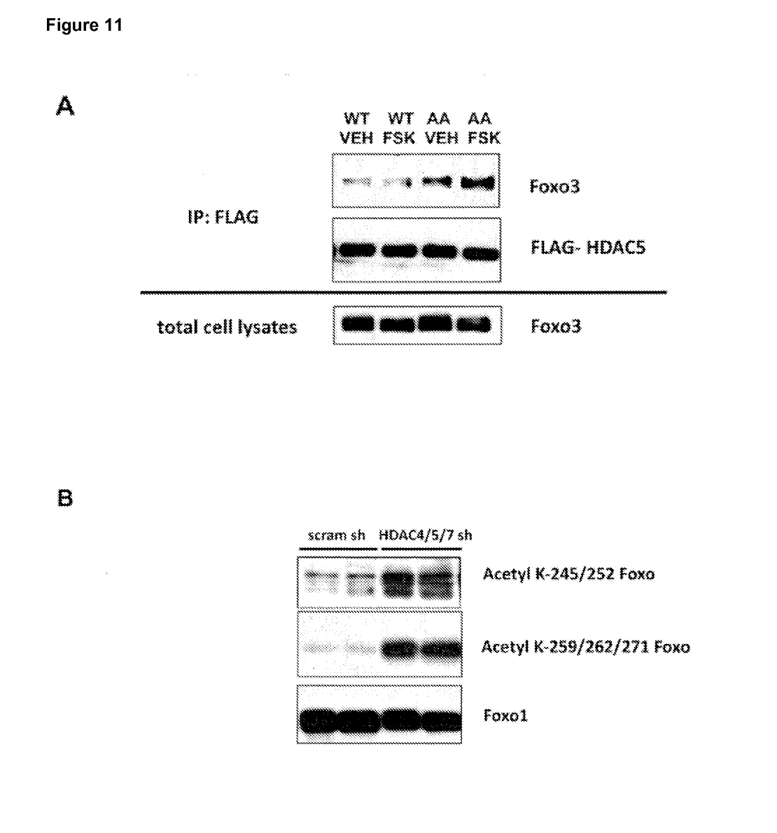

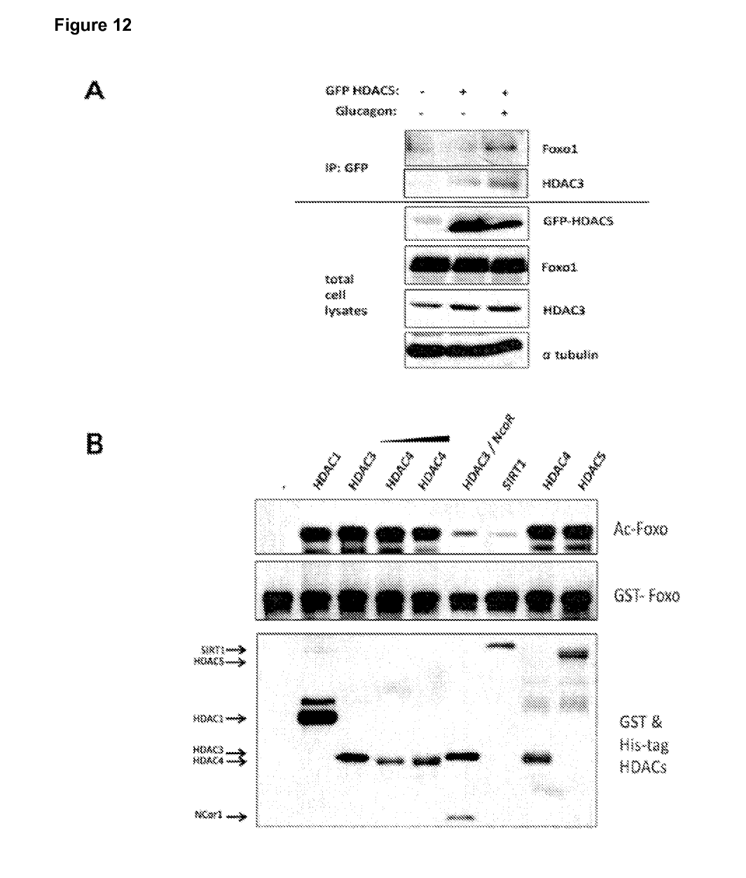

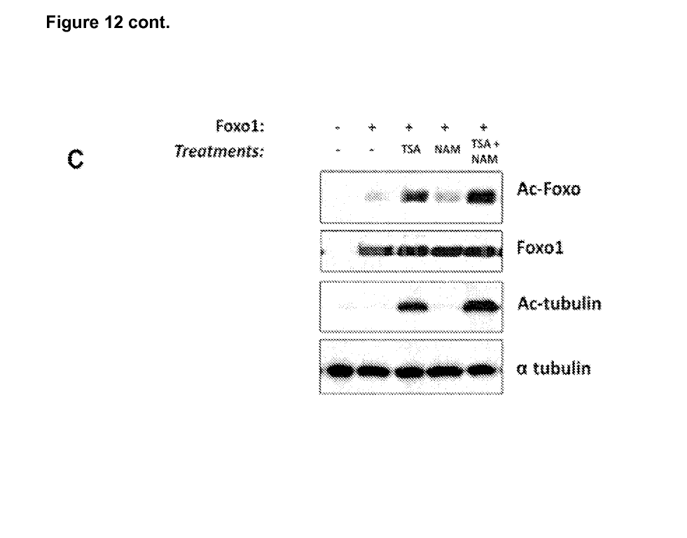

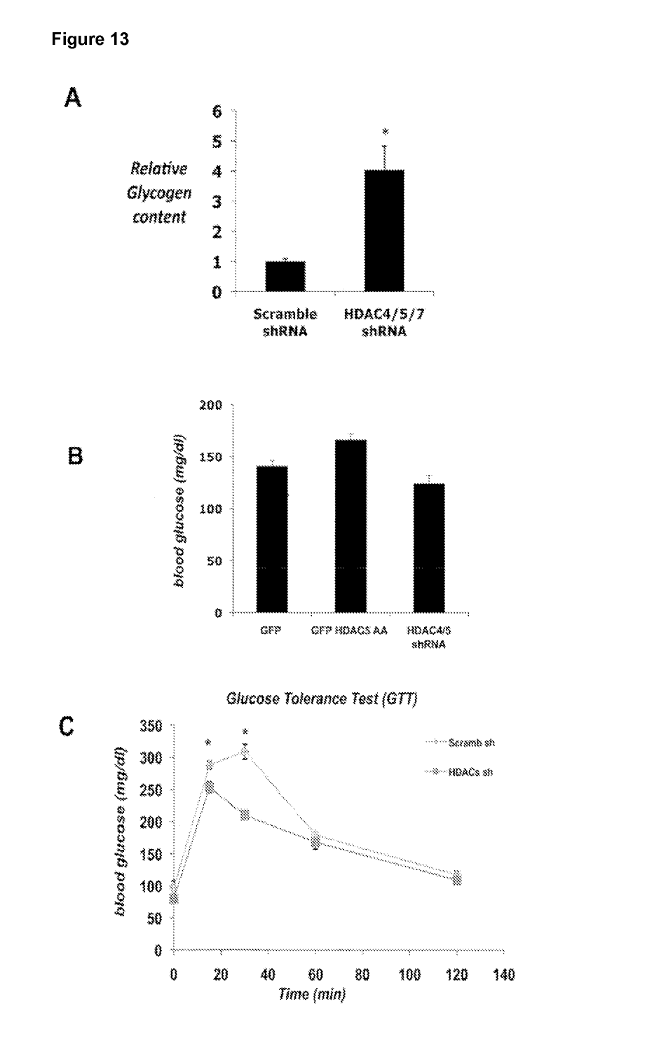

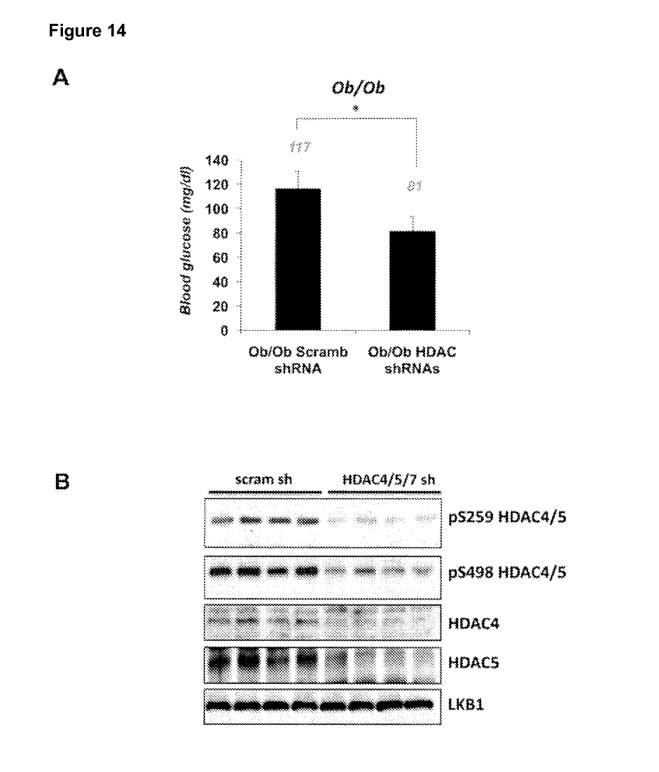

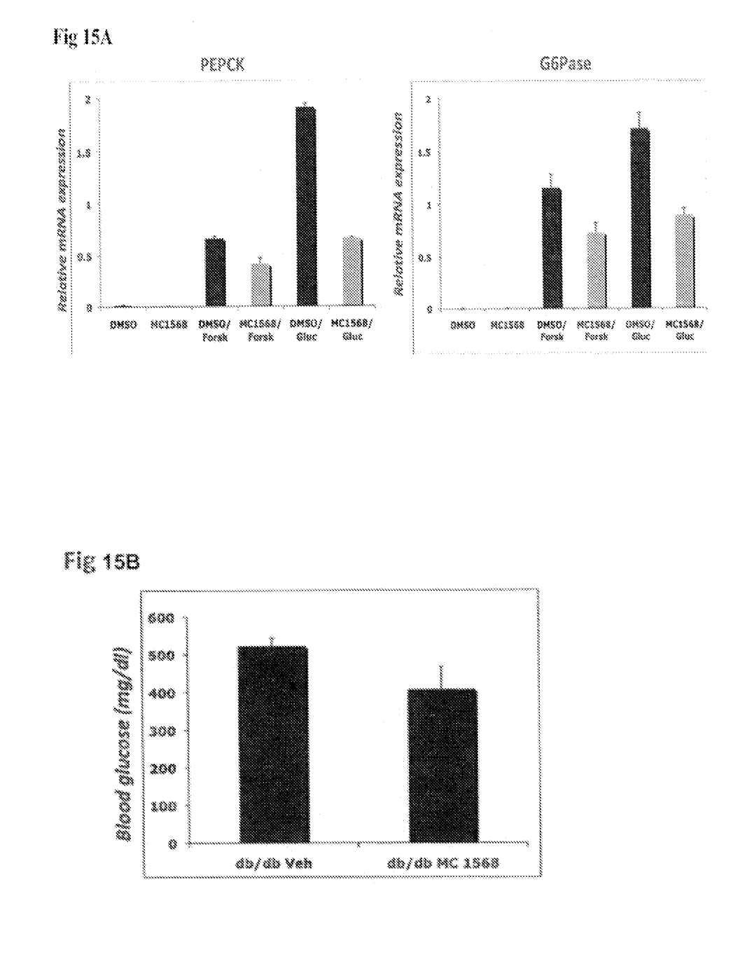

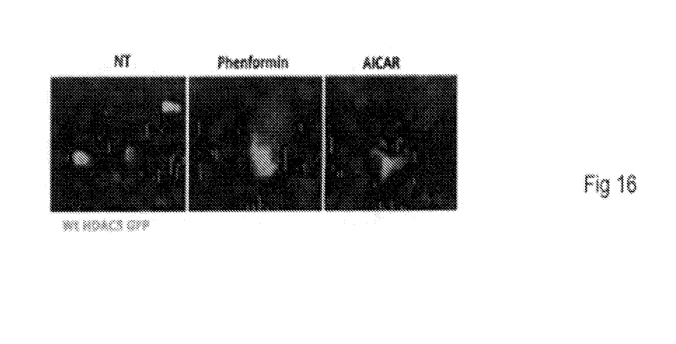

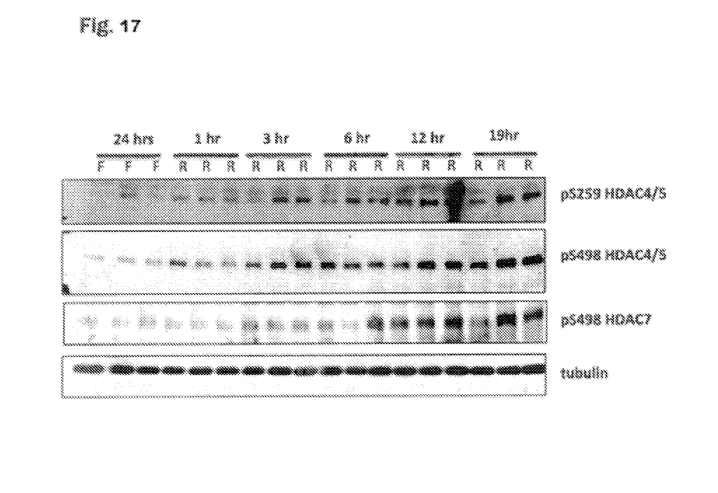

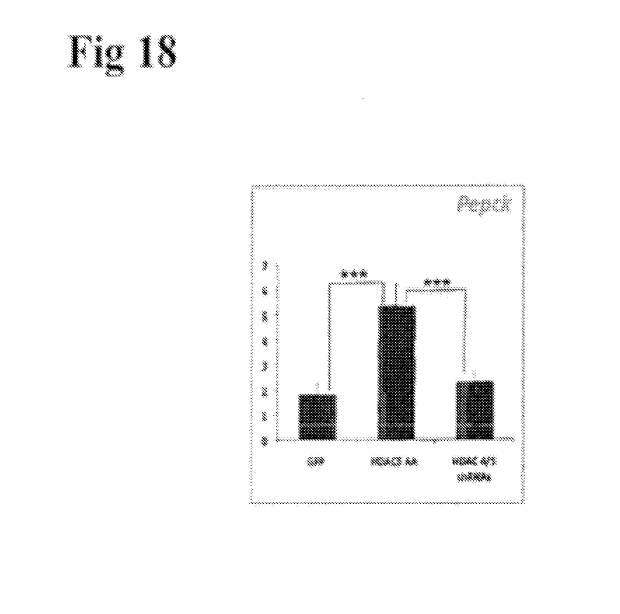

FIG. 8 shows validation of specificity of HDAC antibodies used and assessment of Class IIa HDAC localization and 14-3-3 binding, related to FIG. 1

(A) Hepa1-6 hepatoma, C2C12 myoblasts, or mouse embryonic fibroblasts (MEF) were infected with adenoviruses bearing hairpin shRNAs against murine HDAC4, HDAC5, or HDAC7 as indicated and immunoblotted with indicated antibodies to detect endogenous HDAC proteins.

(B) FLAG-tagged wild-type, Ser259Ala, Ser498Ala, or Ser259Ala/Ser498Ala (AA) mutant HDAC5 constructs were transfected in HEK293T cells, immunoprecipitated with anti-FLAG antibody and immunoblotted with the indicated antibodies.

(C) Activation of endogenous AMPK or overexpression of AMPK relocalizes wild-type (WT) HDAC5-GFP, but not Ser259A/Ser498A (AA)-HDAC5-GFP out of the nucleus. U2OS cells were transfected with wild-type GFP-HDAC5 and then treated with 2 mM AICAR or 1 mM phenformin or vehicle.

(D) U2OS cells were transfected with WT-HDAC5-GFP with or without myc-tagged constitutively active AMPKa2 (1-312) as indicated.

(E) HEK293T cells transfected with indicated myc tagged-AMPKa2 alleles, FLAG-tagged HDAC5 alleles, and GST-14-3-3 and immunoprecipitated with GST-14-3-3 and immunoblotted with indicated antibodies.

(F) Lysates from primary hepatocytes treated with either AMPK agonist A769662 or Vehicle (DMSO) for 1 hr and immunoblotted with indicated antibodies.

FIG. 9 shows that forskolin mimics glucagon dependent effects of de-phosphorylation of the Class IIa HDACs, related to FIG. 2

(A) Mice were starved for 6 h and then treated with saline, glucagon (Gluca) for 45 minutes, or refed for 3 h. Liver lysates were immunoblotted with indicated antibodies.

(B) Primary mouse hepatocytes were treated with vehicle (DMSO) or 10 uM forksolin for indicated times and lysates were immunoblotted with indicated antibodies.

(C) Primary mouse hepatocytes infected with adenovirus expressing GFP-HDAC5 WT were treated with 10 uM forskolin or vehicle (DMSO) for indicated times and imaged

(D) Forskolin-induced WT HDAC5-GFP nuclear localization is similar to Ser259Ala/Ser498Ala HDAC5-GFP constitutive nuclear localization. Primary hepatocytes were infected with adenovirus expressing GFP, WT HDAC5-GFP, or AA-HDAC5-GFP and treated with 10 uM forskolin or vehicle (DMSO) for indicated times and imaged.

FIG. 10 shows glucagon dependent transcriptional effects require Class IIa HDACs, which associate with gluconeogenic promoters, related to FIG. 3

(A) Top 25 genes whose induction by glucagon is attenuated by HDAC4/5 shRNA compared to scrambled control shRNA. Primary hepatocytes were treated with 10 uM Forskolin for 4 h. Ratio reflects comparison of 4 h Forskolin scrambled control shRNA samples over corresponding 4 h forskolin HDAC4/5 shRNA treated samples.

(B) Lysates from primary hepatocytes in parallel cultures to those used in FIG. 3B were immunoblotted with indicated antibodies against endogenous proteins.

(C) HDAC4 antibody but not control IgG immunoprecipitates with the promoter proximal region of the murine G6Pase promoter. Primary hepatocytes were treated with glucagon as indicated then subject to chromatin immunoprecipitation with the indicated antibodies.

(D) HDAC4 ChIP at −209 region of G6Pase locus in cells infected with scrambled (scram) or HDAC4/5/7 shRNA in cells treated with 100 nM glucagon for 1 h where indicated. Data are shown as mean+/−s.e.m. (n=4) * p<0.05, Student's t-test, comparison between Glucagon treated scramble shRNA and Glucagon treated HDAC4/5/7 shRNA samples.

FIG. 11 shows that Class IIa HDACs associate with Foxos and regulate their acetylation on multiple lysines, related to FIG. 4

(A) Primary hepatocytes were transduced with Foxo 3 and Flag-HDAC5 WT or AA adenoviruses and treated with Forskolin (FSK) or Vehicle (VEH) for 1 hr. Lysates were immunoprecipitated for Flag HDAC5 WT or AA and immunoblotted with indicated antibodies.

(B) Assessing acetylation of Foxo 3 with two different Foxo acetylation specific antibodies in primary hepatocytes that are knocked down with either Class IIa HDACs shRNAs or scrambled shRNA.

FIG. 12 shows that Class IIa HDACs associate with Class I HDAC3, which mediates Foxo deacetylation, related to FIG. 5

(A) Primary hepatocytes tranduced with GFP-HDAC5 WT and Glucagon treated were immunoprecipitated with anti-GFP and immunoblotted for endogenous Foxo1 and HDAC3.

(B) In vitro deacetylation assays were performed on recombinant GST-FOXO1, which had been prior acetylated in vitro with a recombinant fragment of p300. GST-FOXO1 acetylation is detected using the Foxo1 K259/262/271 acetylation specific antibody. HDAC3 complexed with Ncor deacetylated GST-FOXO1 as seen in FIG. 4F and not recombinant HDAC4 or HDAC5. Recombinant SIRT1 serves as positive control. Reactions were ran on SDS-PAGE gel and immunoblotted with indicated antibodies.

(C) HEK293T cells were transfected with Myc-Foxo1 WT construct and cells were treated with 1 uM TSA and/or 10 mM NAM for 2 h. Cell lysates were immunoblotted with indicated antibodies.

FIG. 13 shows that loss and gain of function of the Class IIa HDACs in liver augments hepatic glycogen content and blood glucose, related to FIG. 5

(A) Mouse livers knockdowns for Class IIa HDACs or control Scramble shRNA were homogenized and assessed for glycogen content. Data represents the fold change of glycogen content of HDACs shRNA depleted livers compared to control scramble shRNA livers. * Data are shown as mean+/−s.e.m. (n=3). p<0.05, Student's t-test.

(B) Nonphosphorylatable HDAC5 modestly increases blood glucose and HDAC4/5/7 shRNA reduces blood glucose in ad lib fed B6 mice. C57BL/6J mice were tail-vein injected with adenoviruses expressing GFP, AA-HDAC5-GFP, or HDAC4/5 shRNAs and after 4 days, blood glucose was tested.

(C) Glucose tolerance test performed on fasted C57BL/6J mice on normal chow diet tail-vein injected with either scrambled or HDAC4/5/7 shRNA. Data are shown as mean+/−s.e.m. (n=5), * p<0.05, Student's t-test.

FIG. 14 shows that loss of Class IIa HDACs in ob/ob mice reduces fasting blood glucose, related to FIG. 7

(A) Ten week old ob/ob mice were tail-vein injected with adenoviruses bearing either scramble (scram) shRNA or HDAC4/5/7 (HDAC) shRNAs. 5 days later, mice were fasted for 18 h and blood glucose was measured. Average blood glucose value shown in red. Data are shown as mean+/−s.e.m. (n=4). * p<0.001 Student's t-test.

(B) Ob/ob mice were tail-vein injected with adenoviruses bearing indicated shRNAs. Liver lysates were immunoblotted with indicated antibodies.

FIG. 15 shows that suppression of gluconeogenic genes and blood glucose following treatment with the Class II HDAC specific inhibitor MC1568
A. Primary hepatocytes were pre-treated with 5 uM MC1568 (Sigma-Aldrich Catalog #M1824) dissolved in DMSO or DMSO alone (vehicle) for 14 hours and then treated with either vehicle, 10 uM Forskolin or 100 nM Glucagon for 4 hrs. mRNA levels of G6PAse and PEPCK were analyzed by qPCR and normalized to cyclophilin.
B. db/db mice (obesity & type 2 diabetes model from Jackson Laboratory # were intraperitoneally injected once a day with either vehicle [DMSO/sesame oil (20% v/v)] or 50 mg/kg of MC1568 dissolved in vehicle for 4 days. Blood glucose was measured on mice fasted for 22 hours (4 hrs following the final treatment).

FIG. 16 depicts Huh7 cells transfected with GFP-tagged wild-type HDAC5 were treated with 2 mM phenformin or 2 mM AICAR for 1 h and visualized for GFP.

FIG. 17 shows that HDAC4/5/7 phosphorylation is reduced with fasting. Liver lysates were isolated from B6 mice fasted for 24 h, then refed and harvested at times indicated and immunoblotted with indicated antibodies.

FIG. 18 depicts Q-PCR for PEPCK from livers of 18 h fasted B6 mice tail-vein injected 5 days prior with GFP or GFP-HDAC5-AA or HDAC4/5 shRNAs as indicated. Data are shown as mean+/−s.e.m. (n=6). * p<0.01 ** p<0.001 ***p<0.0001 Unpaired student's t-test.

### DETAILED DESCRIPTION OF THE INVENTION

Class IIa histone deacetylases (HDACs) are signal-dependent modulators of transcription with established roles in muscle differentiation and neuronal survival. Applicants show herein that in liver, Class IIa HDACs (e.g., HDAC4, 5, and 7) are phosphorylated and excluded from the nucleus by AMPK family kinases. In response to the fasting hormone glucagon, Class IIa HDACs are rapidly dephosphorylated and translocated to the nucleus where they associate with the promoters of gluconeogenic enzymes such as G6Pase. In turn, HDAC4/5 recruit HDAC3, which results in the acute transcriptional induction of these genes via deacetylation and activation of Foxo family transcription factors. Loss of Class IIa HDACs in murine liver results in inhibition of FOXO target genes and lowers blood glucose, resulting in increased glycogen storage. In addition, suppression of Class IIa HDACs in mouse models of Type 2 Diabetes ameliorates hyperglycemia. Accordingly, in certain embodiments, the instant invention relates to inhibitors of Class I/II HDACs as therapeutics for metabolic syndrome.

Applicants show herein that phosphorylation of Class IIa HDACs is controlled in liver by LKB1-dependent kinases, but in response to glucagon, Class IIa HDACs are rapidly dephosphorylated and translocate to the nucleus where they associate with the G6pc and Pck1 promoters. Glucagon is known to stimulate expression of these genes in hepatocytes through PKA-mediated effects on CREB (Montminy et al., 2004), and through effects on FOXO of an unknown mechanism (Matsumoto et al., 2007). Applicants demonstrate that Class IIa HDACs recruit HDAC3 to gluconeogenic loci and regulate FOXO acetylation in hepatocytes and liver. Knockdown of Class IIa HDACs results in FOXO hyperacetylation, loss of FOXO target genes, and reduction of hyperglycemia in several mouse models of type diabetes, indicating that these proteins play key roles in mammalian glucose homeostasis.

Applicants show that Class IIa HDACs are critical components of the transcriptional response to fasting in liver, shuttling into the nucleus in response to glucagon. Once nuclear, they bind to the promoters of gluconeogenic target genes and mediate their transcriptional induction. In certain embodiments, they mediate transcriptional induction through promoting deacetylation and activation of Foxo transcription factors (FIG. 7F). These findings illuminate a mechanism by which glucagon can acutely stimulate FOXO activity, providing a molecular basis for how FOXO mediates effects of both fasting hormones and insulin on hepatic glucose production (Matsumoto et al., 2007). Consistent with this, hepatic knockdown of Class IIa HDACs in vivo results in lowered blood glucose and altered glycogen storage, phenocopying hepatic deficiency of Foxo1 in mice (Matsumoto et al., 2007), as well as the G6pc deficiency in mice and human Glycogen Storage Disease Type I (GSDI) patients (Salganik et al. 2009; Peng et al., 2009).

Thus, fasting may promote FOXO activation by a two-pronged mechanism where loss of insulin signaling results in dephosphorylation of the Akt sites in FOXO, allowing its re-entry into the nucleus, while glucagon-induced dephosphorylation of the Class IIa HDACs results in their nuclear translocation and deacetylation of nuclear FOXO, enhancing FOXO DNA-binding activity and association with gluconeogenic gene promoters. Class IIa HDACs are appreciated for roles in transcriptional repression of muscle differentiation through modulation of the Mef2 family of transcription factors (Haberland et al., 2009). FOXO family members have also been shown to work in concert with MEF2 family members in cardiomyocytes (Creemers et al., 2006) and the only transcription factor that HDAC3 has been previously reported to deacetylate is Mef2 itself (Gregoire et al., 2007). Without being bound to theory, Applicants believe there may be a possible coordinated regulation of FOXO and MEF2 by a Class IIa HDAC HDAC3 deacetylase complex. In certain embodiments, the invention relates to additional non-histone targets whose acetylation is controlled by Class IIa HDACs.

FOXO has also been previously shown to be a target of SIRT1 in a number of cell types, particularly defined in muscle (Canto et al., 2009). Like shown herein for Class IIa HDACs, SIRT1 activity in liver is also thought to be increased following fasting. It is notable, however, that in previous reports, SIRT1 levels are not increased rapidly following fasting (Rodgers et al., 2005), though it is possible that SIRT1 may also be controlled post-translationally as well. SIRT1 has been shown to control gluconeogenesis and other hepatic processes though a number of downstream targets (reviewed in Houtkooper et al., 2010).

Without being bound to theory, Applicants believe that both the CRTC family of co-activators and the Class IIa HDACs may be coordinately regulated in liver by the opposing activity of LKB1-dependent kinases stimulating 14-3-3 docking and cytoplasmic sequestration, and glucagon-induced signals promoting de-phosphorylation and nuclear import. PKA has been demonstrated to directly phosphorylate and inhibit AMPK, SIK1, and SIK2 (Screaton et al., 2004; Hurley et al., 2006; Berdeaux et al., 2007; Djouder et al., 2010). Thus, cAMP induction by glucagon should block several LKB1-dependent kinases from phosphorylating the Class IIa HDACs. In some embodiments, PKA may actively stimulate a phosphatase such as calcineurin, and this achieves the efficient nuclear translocation of CRTC and HDAC proteins in parallel. By promoting the simultaneous activation of a positive regulator of CREB-dependent transcription (CRTCs) and a positive regulator of FOXO-dependent transcription (HDAC4/5/7), glucagon further promotes the expression of target genes bearing CREB and FOXO responsive elements including the gluconeogenic enzymes.

As Applicants show that AMPK activation by metformin treatment leads to increased HDAC4/5/7 phosphorylation and inactivation, this provides another mechanism by which the widely used type 2 diabetes therapeutic serves to suppress hepatic gluconeogenesis and lower blood glucose (Shaw et al., 2005). Perhaps most unexpectedly, the results described herein indicate that Class I and Class IIa HDACs in the liver of type 2 diabetic rodent models actively contribute to the hyperglycemic phenotype of these animals, which may result from a strong role for FOXO in hyperglycemia in these insulin resistant states. Remarkably, shRNA-mediated suppression of Class IIa HDAC function led to a dramatic reduction of blood glucose levels in LKB1 liver-specific knockout mice, high fat diet mice, db/db mice, and ob/ob mice. Accordingly, in certain embodiments, small molecules that inhibit Class I/IIa HDACs may be useful as diabetes therapeutics, for example, when extended to human studies. Given the intense ongoing effort in the pharmaceutical industry to develop HDAC inhibitors as anti-cancer agents (Witt et al., 2009), their use for the treatment of metabolic disease would be an important utility.

As Class IIa HDACs are activated through hormonal increases in cAMP, this may augment or complement the conditions in which the NAD+-dependent sirtuins may act on FOXO in different tissues (Brunet et al., 2004; Motta et al., 2004; van der Horst et al., 2004). SIRT1 has been shown to contribute to transcriptional control of glucose and lipid metabolism through the deacetylation of FOXO, PGC1a, HNF4a, SREBP1, STAT3, and LXR amongst other targets (Feige and Auwerx, 2008). Expectantly, hepatic delivery of SIRT1 shRNA has been shown to reduce gluconeogenesis (Rodgers and Puigserver, 2007), though the effect of loss of SIRT1 function in mice on glucose homeostasis varies in different studies (Rodgers and Puigserver, 2007; Chen et al., 2008; Purushotham et al., 2009), which may be due to the strain background of the mice used, the method of SIRT1 depletion, and/or the potential compensation of other sirtuins for loss of SIRT1 function in this process. Extensive crosstalk is likely to exist between these different metabolic regulatory systems. For example in muscle, calcium-dependent increases in AMPK activity result in increased SIRT1 activity following exercise (Canto et al., 2009).

In certain embodiments, small molecules or other compounds that specifically inhibit one or more Class IIa HDACs are useful as diabetes therapeutics, such as type 1 diabetes, type 2 diabetes, gestational diabetes, and MODY (maturity onset diabetes of the young) diabetes therapeutics. In certain embodiments, small molecules or other compounds that specifically inhibit one or more Class IIa HDACs are useful in the treatment of other metabolic disorders, such as insulin resistance, obesity, metabolic syndrome, hyperglycemia, and impaired glucose tolerance. The terms “metabolic disorder” and “metabolic disease” are used interchangeably herein and typically refer to a disorder characterized by one or more problems with an organism's metabolism. Examples of metabolic disorders include, without limitation, diabetes, insulin resistance, obesity, metabolic syndrome, hyperglycemia, and impaired glucose tolerance.

In yet other embodiments, inhibitors specific to Class IIa HDACs are useful in the treatment of medical conditions characterized by one or more hyperactivated FOXO transcription factors. Examples of such medical conditions include muscle-wasting diseases such as cachexia, muscle wasting, age-related sarcopenia, and muscular dystrophy.

Almost all enzymes known to regulate FOXO transcription factors do so by reducing FOXO transcription factor activity. Therefore, inhibiting these enzymes leads to increased FOXO activity, potentially leading to the development of medical conditions such as muscle wasting and diabetes. By contrast, the instant invention provides methods of inhibiting FOXO transcription factor activity, by administering to a subject in need thereof an inhibitor specific to a Class IIa HDAC.

An example of a Class IIa-specific HDAC inhibitor is MC1568, which is manufactured by Sigma-Aldrich (catalogue # M1824). As described herein, MC1568 lowers blood glucose in a type 2 diabetic non-primate animal model (db/db mice) as well as suppresses the expression of the two gluconeogenic genes PEPCK and G6P (see, for example, FIG. 15). In certain embodiments, an inhibitor of one or more Class IIa HDACs is a pharmaceutically acceptable salt of MC1568.

As used herein, the terms “drug,” “agent,” and “compound” encompass any composition of matter or mixture which provides some pharmacologic effect that can be demonstrated in-vivo or in vitro. This includes small molecules, nucleic acids, antibodies, microbiologicals, vaccines, vitamins, and other beneficial agents. As used herein, the terms further include any physiologically or pharmacologically active substance that produces a localized or systemic effect in a patient.

The term “nucleic acid” encompasses DNA, RNA (e.g., mRNA, tRNA), heteroduplexes, and synthetic molecules capable of encoding a polypeptide and includes all analogs and backbone substitutes such as PNA that one of ordinary skill in the art would recognize as capable of substituting for naturally occurring nucleotides and backbones thereof. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence.

Unless otherwise indicated, nucleic acids are written left to right in 5′ to 3′ orientation; amino acid sequences are written left to right in amino to carboxy orientation, respectively.

As used herein, the term “amino acid sequence” is synonymous with the terms “polypeptide,” “protein,” and “peptide,” and are used interchangeably. The conventional one-letter or three-letter code for amino acid residues are used herein.

As used herein, a “synthetic” molecule is produced by in vitro chemical or enzymatic synthesis rather than by an organism.

As used herein, the term “expression” refers to the process by which a polypeptide is produced based on the nucleic acid sequence of a gene. The process includes both transcription and translation.

A “gene” refers to the DNA segment encoding a polypeptide or RNA.

By “homolog” is meant an entity having a certain degree of identity with the subject amino acid sequences and the subject nucleotide sequences. As used herein, the term “homolog” covers identity with respect to structure and/or function, for example, the expression product of the resultant nucleotide sequence has the enzymatic activity of a subject amino acid sequence. With respect to sequence identity, preferably there is at least 70%, 75%, 80%, 81%, 82%, 83%, 84%, 85%, 86%, 87%, 88%, 89%, 90%, 91%, 92%, 93%, 94%, 95%, 96%, 97%, 98%, or even 99% sequence identity. These terms also encompass allelic variations of the sequences. The term, homolog, may apply to the relationship between genes separated by the event of speciation or to the relationship between genes separated by the event of genetic duplication.

Thus, in certain embodiments, the present invention encompasses the use of variants, homologues and derivatives of a Class IIa HDAC nucleic acid and/or amino acid sequence. Examples of Class IIa HDAC nucleic acid sequences include the human Class IIa HDAC nucleic acid sequences available through the National Center for Biotechnology Information (NCBI) website, such as GenBank Nos. NM_{—}006037 (HDAC4); NM_{—}001015053 (HDAC5, transcript variant 3), NM_{—}005474 (HDAC5, transcript variant 1); NM_{—}001098416 (HDAC7, transcript variant 4), NM_{—}015401 (HDAC7, transcript variant 1); and NM_{—}058176 (HDAC9, transcript variant 1) and their corresponding amino acid sequences. In one embodiment, the sequences, such as variants, homologs and derivatives of a human Class IIa HDAC amino acid sequence, may also have deletions, insertions or substitutions of amino acid residues which produce a silent change and result in a functionally equivalent substance.

Relative sequence identity can be determined by commercially available computer programs that can calculate % identity between two or more sequences using any suitable algorithm for determining identity, using, for example, default parameters. A typical example of such a computer program is CLUSTAL. Advantageously, the BLAST algorithm is employed, with parameters set to default values. The BLAST algorithm is described in detail on the NCBI website.

The homologs of the peptides as provided herein typically have structural similarity with such peptides. A homolog of a polypeptide includes one or more conservative amino acid substitutions, which may be selected from the same or different members of the class to which the amino acid belongs.

In one embodiment, the sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The present invention also encompasses conservative substitution (substitution and replacement are both used herein to mean the interchange of an existing amino acid residue with an alternative residue) that may occur e.g., like-for-like substitution such as basic for basic, acidic for acidic, polar for polar, etc. Non-conservative substitution may also occur e.g., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine. Conservative substitutions that may be made are, for example, within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxyl amino acids (Serine, Threonine), large amino acids (Phenylalanine and Tryptophan) and small amino acids (Glycine, Alanine).

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Many methods of sequencing genomic DNA are known in the art, and any such method can be used, see for example Sambrook et al., Molecular Cloning; A Laboratory Manual 2d ed. (1989). For example, a DNA fragment of interest can be amplified using the polymerase chain reaction or some other cyclic polymerase mediated amplification reaction. The amplified region of DNA can then be sequenced using any method known in the art. Advantageously, the nucleic acid sequencing is by automated methods (reviewed by Meldrum, Genome Res. September 2000; 10(9):1288-303, the disclosure of which is incorporated by reference in its entirety), for example using a Beckman CEQ 8000 Genetic Analysis System (Beckman Coulter Instruments, Inc.). Methods for sequencing nucleic acids include, but are not limited to, automated fluorescent DNA sequencing (see, e.g., Watts & MacBeath, Methods Mol Biol. 2001; 167:153-70 and MacBeath et al., Methods Mol Biol. 2001; 167:119-52), capillary electrophoresis (see, e.g., Bosserhoff et al., Comb Chem High Throughput Screen. December 2000; 3(6):455-66), DNA sequencing chips (see, e.g., Jain, Pharmacogenomics. August 2000; 1(3):289-307), mass spectrometry (see, e.g., Yates, Trends Genet. January 2000; 16(1):5-8), pyrosequencing (see, e.g., Ronaghi, Genome Res. January 2001; 11(1):3-11), and ultrathin-layer gel electrophoresis (see, e.g., Guttman & Ronai, Electrophoresis. December 2000; 21 (18):3952-64), the disclosures of which are hereby incorporated by reference in their entireties. The sequencing can also be done by any commercial company. Examples of such companies include, but are not limited to, the University of Georgia Molecular Genetics Instrumentation Facility (Athens, Ga.) or SeqWright DNA Technologies Services (Houston, Tex.).

Any one of the methods known in the art for amplification of DNA may be used, such as for example, the polymerase chain reaction (PCR), the ligase chain reaction (LCR) (Barany, F., Proc. Natl. Acad. Sci. (U.S.A.) 88:189-193 (1991)), the strand displacement assay (SDA), or the oligonucleotide ligation assay (“OLA”) (Landegren, U. et al., Science 241:1077-1080 (1988)). Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990)). Other known nucleic acid amplification procedures, such as transcription-based amplification systems (Malek, L. T. et al., U.S. Pat. No. 5,130,238; Davey, C. et al., European Patent Application 329,822; Schuster et al., U.S. Pat. No. 5,169,766; Miller, H. I. et al., PCT Application WO89/06700; Kwoh, D. et al., Proc. Natl. Acad. Sci. (U.S.A.) 86:1173 (1989); Gingeras, T. R. et al., PCT Application W088/10315)), or isothermal amplification methods (Walker, G. T. et al., Proc. Natl. Acad. Sci. (U.S.A.) 89:392-396 (1992)) may also be used.

To perform a cyclic polymerase mediated amplification reaction according to the present invention, the primers are hybridized or annealed to opposite strands of the target DNA, the temperature is then raised to permit the thermostable DNA polymerase to extend the primers and thus replicate the specific segment of DNA spanning the region between the two primers. Then the reaction is thermocycled so that at each cycle the amount of DNA representing the sequences between the two primers is doubled, and specific amplification of gene DNA sequences, if present, results.

Any of a variety of polymerases can be used in the present invention. For thermocyclic reactions, the polymerases are thermostable polymerases such as Taq, KlenTaq, Stoffel Fragment, Deep Vent, Tth, Pfu, Vent, and UlTma, each of which are readily available from commercial sources. For non-thermocyclic reactions, and in certain thermocyclic reactions, the polymerase will often be one of many polymerases commonly used in the field, and commercially available, such as DNA pol 1, Klenow fragment, T7 DNA polymerase, and T4 DNA polymerase. Guidance for the use of such polymerases can readily be found in product literature and in general molecular biology guides.

Typically, the annealing of the primers to the target DNA sequence is carried out for about 2 minutes at about 37-55° C., extension of the primer sequence by the polymerase enzyme (such as Taq polymerase) in the presence of nucleoside triphosphates is carried out for about 3 minutes at about 70-75° C., and the denaturing step to release the extended primer is carried out for about 1 minute at about 90-95° C. However, these parameters can be varied, and one of skill in the art would readily know how to adjust the temperature and time parameters of the reaction to achieve the desired results. For example, cycles may be as short as 10, 8, 6, 5, 4.5, 4, 2, 1, 0.5 minutes or less.

Also, “two temperature” techniques can be used where the annealing and extension steps may both be carried out at the same temperature, typically between about 60-65° C., thus reducing the length of each amplification cycle and resulting in a shorter assay time.

Typically, the reactions described herein are repeated until a detectable amount of product is generated. Often, such detectable amounts of product are between about 10 ng and about 100 ng, although larger quantities, e.g. 200 ng, 500 ng, 1 mg or more can also, of course, be detected. In terms of concentration, the amount of detectable product can be from about 0.01 pmol, 0.1 pmol, 1 pmol, 10 pmol, or more. Thus, the number of cycles of the reaction that are performed can be varied, the more cycles are performed, the more amplified product is produced. In certain embodiments, the reaction comprises 2, 5, 10, 15, 20, 30, 40, 50, or more cycles.

For example, the PCR reaction may be carried out using about 25-50 μl samples containing about 0.01 to 1.0 ng of template amplification sequence, about 10 to 100 pmol of each generic primer, about 1.5 units of Taq DNA polymerase (Promega Corp.), about 0.2 mM dDATP, about 0.2 mM dCTP, about 0.2 mM dGTP, about 0.2 mM dTTP, about 15 mM MgCl.sub.2, about 10 mM Tris-HCl (pH 9.0), about 50 mM KCl, about 1 μg/ml gelatin, and about 10 μl/ml Triton X-100 (Saiki, 1988).

Those of ordinary skill in the art are aware of the variety of nucleotides available for use in the cyclic polymerase mediated reactions. Typically, the nucleotides will consist at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as chain-terminating nucleotides, dideoxynucleotides and boronated nuclease-resistant nucleotides. Commercial kits containing the reagents most typically used for these methods of DNA sequencing are available and widely used. Other nucleotide analogs include nucleotides with bromo-, iodo-, or other modifying groups, which affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them.

In certain embodiments, oligonucleotides that can be used as primers to amplify specific nucleic acid sequences of a gene in cyclic polymerase-mediated amplification reactions, such as PCR reactions, consist of oligonucleotide fragments. Such fragments should be of sufficient length to enable specific annealing or hybridization to the nucleic acid sample. The sequences typically will be about 8 to about 44 nucleotides in length, but may be longer. Longer sequences, e.g., from about 14 to about 50, are advantageous for certain embodiments.

In embodiments where it is desired to amplify a fragment of DNA, primers having contiguous stretches of about 8, 9, 10, 11, 12, 13, 14, 15, 16, 17, 18, 19, 20, 21, 22, 23, or 24 nucleotides from a gene sequence are contemplated.

As used herein, “hybridization” refers to the process by which one strand of nucleic acid base pairs with a complementary strand, as occurs during blot hybridization techniques and PCR techniques.

Whichever probe sequences and hybridization methods are used, one ordinarily skilled in the art can readily determine suitable hybridization conditions, such as temperature and chemical conditions. Such hybridization methods are well known in the art. For example, for applications requiring high selectivity, one will typically desire to employ relatively stringent conditions for the hybridization reactions, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand. It is generally appreciated that conditions can be rendered more stringent by the addition of increasing amounts of formamide. Other variations in hybridization reaction conditions are well known in the art (see for example, Sambrook et al., Molecular Cloning; A Laboratory Manual 2d ed. (1989)).

Hybridization conditions are based on the melting temperature (Tm) of the nucleic acid binding complex, as taught, e.g., in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of ordinary skill in the art, a maximum stringency hybridization can be used to identify or detect identical nucleotide sequences while an intermediate (or low) stringency hybridization can be used to identify or detect similar or related polynucleotide sequences.

In one aspect, the present invention employs nucleotide sequences that can hybridize to another nucleotide sequence under stringent conditions (e.g., 65° C. and 0.1×SSC {1×SSC=0.15 M NaCl, 0.015 M Na3 Citrate pH 7.0). Where the nucleotide sequence is double-stranded, both strands of the duplex, either individually or in combination, may be employed by the present invention. Where the nucleotide sequence is single-stranded, it is to be understood that the complementary sequence of that nucleotide sequence is also included within the scope of the present invention.

Stringency of hybridization refers to conditions under which polynucleic acid hybrids are stable. Such conditions are evident to those of ordinary skill in the field. As known to those of ordinary skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridization reaction is performed under conditions of higher stringency, followed by washes of varying stringency.

As used herein, high stringency includes conditions that permit hybridization of only those nucleic acid sequences that form stable hybrids in 1 M Na+ at 65-68° C. High stringency conditions can be provided, for example, by hybridization in an aqueous solution containing 6×SSC, 5×Denhardt's, 1% SDS (sodium dodecyl sulphate), 0.1 Na+ pyrophosphate and 0.1 mg/ml denatured salmon sperm DNA as non-specific competitor. Following hybridization, high stringency washing may be done in several steps, with a final wash (about 30 minutes) at the hybridization temperature in 0.2-0.1×SSC, 0.1% SDS.

It is understood that these conditions may be adapted and duplicated using a variety of buffers, e.g., formamide-based buffers, and temperatures. Denhardt's solution and SSC are well known to those of ordinary skill in the art as are other suitable hybridization buffers (see, e.g., Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridization conditions are typically determined empirically, as the length and the GC content of the hybridizing pair also play a role.

Nucleic acid molecules that differ from the sequences of the primers and probes disclosed herein, are intended to be within the scope of the invention. Nucleic acid sequences that are complementary to these sequences, or that are hybridizable to the sequences described herein under conditions of standard or stringent hybridization, and also analogs and derivatives are also intended to be within the scope of the invention. Advantageously, such variations will differ from the sequences described herein by only a small number of nucleotides, for example by 1, 2, or 3 nucleotides.

Nucleic acid molecules corresponding to natural allelic variants, homologues (i.e., nucleic acids derived from other species), or other related sequences (e.g., paralogs) of the sequences described herein can be isolated based on their homology to the nucleic acids disclosed herein, for example by performing standard or stringent hybridization reactions using all or a portion of the known sequences as probes. Such methods for nucleic acid hybridization and cloning are well known in the art.

Similarly, a nucleic acid molecule detected in the methods of the invention may include only a fragment of the specific sequences described. Fragments provided herein are defined as sequences of at least 6 (contiguous) nucleic acids, a length sufficient to allow for specific hybridization of nucleic acid primers or probes, and are at most some portion less than a full-length sequence. Fragments may be derived from any contiguous portion of a nucleic acid sequence of choice. Derivatives and analogs may be full length or other than full length, if the derivative or analog contains a modified nucleic acid or amino acid, as described below.

Derivatives, analogs, homologues, and variants of the nucleic acids of the invention include, but are not limited to, molecules comprising regions that are substantially homologous to the nucleic acids of the invention, in various embodiments, by at least about 70%, 80%, 85%, 90%, 95%, 96%, 97%, 98%, or even 99% identity over a nucleic acid sequence of identical size or when compared to an aligned sequence in which the alignment is done by a computer homology program known in the art.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/−N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N N_{ref}=8; N N_{dif}=2). “Homology” or “identity” can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences. Without undue experimentation, the ordinarily skilled artisan can consult with many other programs or references for determining percent homology. “Antisense” nucleic acids are DNA or RNA molecules that are complementary to at least a portion of a specific mRNA molecule (Weintraub, Scientific American 262 40, 1990). In the cell, the antisense nucleic acids hybridize to the corresponding mRNA, forming a double-stranded molecule. This interferes with the translation of the mRNA since the cell will not translate an mRNA that is double-stranded. Antisense oligomers of at least about 15, about 20, about 25, about 30, about 35, about 40, or of at least about 50 nucleotides are preferred, since they are easily synthesized and are less likely to cause non-specific interference with translation than larger molecules. The use of antisense methods to inhibit the in vitro translation of genes is well known in the art (Marcus-Sakura Anal. Biochem. 172: 289, 1998).

The invention provides for nucleic acids complementary to (e.g., antisense sequences to) cellular modulators of Class IIa HDAC activity. Antisense sequences are capable of inhibiting the transport, splicing or transcription of protein-encoding genes. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind gene or message, in either case preventing or inhibiting the production or function of the protein. The association can be through sequence specific hybridization. Another useful class of inhibitors includes oligonucleotides that cause inactivation or cleavage of protein message. The oligonucleotide can have enzyme activity which causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

Short double-stranded RNAs (dsRNAs; typically <30 nucleotides) can be used to silence the expression of target genes in animals and animal cells. Upon introduction, the long dsRNAs enter the RNA interference (RNAi) pathway which involves the production of shorter (20-25 nucleotide) small interfering RNAs (siRNAs) and assembly of the siRNAs into RNA-induced silencing complexes (RISCs). The siRNA strands are then unwound to form activated RISCs, which cleave the target RNA. Double stranded RNA has been shown to be extremely effective in silencing a target RNA. Introduction of double stranded RNA corresponding to, e.g., a Class IIa HDAC gene, would be expected to modify the Class IIa HDAC-related functions discussed herein.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript. The term “RNAi” stands for RNA interference. This term is understood in the art to encompass technology using RNA molecules that can silence genes. See, for example, McManus, et al. Nature Reviews Genetics 3: 737, 2002. In this application, the term “RNAi” encompasses molecules such as short interfering RNA (siRNA), small hairpin or short hairpin RNA (shRNA), microRNAs, and small temporal RNA (stRNA). Generally speaking, RNA interference results from the interaction of double-stranded RNA with genes.

“Small interfering RNA” (siRNA) refers to double-stranded RNA molecules from about 10 to about 30 nucleotides long that are named for their ability to specifically interfere with protein expression through RNA interference (RNAi). Preferably, siRNA molecules are 12-28 nucleotides long, more preferably 15-25 nucleotides long, still more preferably 19-23 nucleotides long and most preferably 21-23 nucleotides long. Therefore, preferred siRNA molecules are 12, 13, 14, 15, 16, 17, 18, 19, 20, 21, 22, 23, 24, 25, 26, 27 28 or 29 nucleotides in length.

RNAi is a two-step mechanism (Elbashir et al., Genes Dev., 15: 188-200, 2001). First, long dsRNAs are cleaved by an enzyme known as Dicer in 21-23 ribonucleotide (nt) fragments, called small interfering RNAs (siRNAs). Then, siRNAs associate with a ribonuclease complex (termed RISC for RNA Induced Silencing Complex) which target this complex to complementary mRNAs. RISC then cleaves the targeted mRNAs opposite the complementary siRNA, which makes the mRNA susceptible to other RNA degradation pathways.

siRNAs of the present invention are designed to interact with a target ribonucleotide sequence, meaning they complement a target sequence sufficiently to bind to the target sequence. The present invention also includes siRNA molecules that have been chemically modified to confer increased stability against nuclease degradation, but retain the ability to bind to target nucleic acids that may be present.

The invention provides antisense oligonucleotides capable of binding messenger RNA, e.g., mRNA encoding Class IIa HDAC4, that can inhibit polypeptide activity by targeting mRNA. Strategies for designing antisense oligonucleotides are well described in the scientific and patent literature, and the ordinarily skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are typically used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can also be used. For example, peptide nucleic acids (PNAs) containing non-ionic backbones, such as N-(2-aminoethyl) glycine units can be used. Antisense oligonucleotides having phosphorothioate linkages can also be used, as described in Mata, Toxicol Appl Pharmacol. 144: 189-197, 1997; Antisense Therapeutics, ed. Agrawal, Humana Press, Totowa, N.J., 1996. Antisense oligonucleotides having synthetic DNA backbone analogues can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold, J. of Biol. Chem. 270: 13581-13584, 1995).

Knockdown of Class IIa HDACs results in FOXO hyperacetylation, loss of FOXO target genes, and reduction of hyperglycemia in several mouse models of type diabetes, indicating that these proteins play key roles in mammalian glucose homeostasis. In certain embodiments, the invention relates to animals that have at least one modulated Class IIa HDAC function. Such modulated functions include, among others, altered gluconeogenesis. The ordinarily skilled artisan will also recognize that alterations in an animal's ability to regulate gluconeogenesis may be assessed by various assays, including by way of example, by assessing changes in expression or activity of molecules involved in gluconeogenesis for example, by measuring expression of FOXO target genes and/or protein expression and/or activity levels of specific fasting response proteins (e.g., proteins induced in response to glucagon stimulation).

Animals having a modified Class IIa HDAC-related function include transgenic animals showing an altered gluconeogenesis due to transformation with constructs using antisense or siRNA technology that affect transcription or expression from a Class IIa HDAC gene. Such animals exhibit an altered glucose homeostasis, such as, for example, a reduction in glucose levels.

Accordingly, in another series of embodiments, the present invention provides methods of screening or identifying proteins, small molecules or other compounds which are capable of inducing or inhibiting the activity or expression of Class IIa HDAC genes and proteins. The assays may be performed, by way of example, in vitro using transformed or non-transformed cells, immortalized cell lines, or in vivo using transformed animal models enabled herein.

To aid in the detection of a protein or nucleic acid, labels are typically used—such as any readily detectable reporter, for example, a fluorescent, bioluminescent, phosphorescent, radioactive, etc. reporter. For example, labels suitable for use in the methods and compositions of the instant invention include green fluorescent protein, yellow fluorescent protein, blue fluorescent protein, and red fluorescent protein. Examples of such reporters (e.g., green fluorescent protein, red fluorescent protein), their detection, coupling to targets/probes, etc. are disclosed herein, for example, in the non-limiting examples.

The present invention further contemplates direct and indirect labelling techniques. For example, direct labelling includes incorporating fluorescent dyes directly into a nucleotide sequence (e.g., dyes are incorporated into nucleotide sequence by enzymatic synthesis in the presence of labelled nucleotides or PCR primers). Direct labelling schemes include using families of fluorescent dyes with similar chemical structures and characteristics. In certain embodiments comprising direct labelling of nucleic acids, cyanine or alexa analogs are utilized. In other embodiments, indirect labelling schemes can be utilized, for example, involving one or more staining procedures and reagents that are used to label a protein in a protein complex (e.g., a fluorescent molecule that binds to an epitope on a protein in the complex, thereby providing a fluorescent signal by virtue of the conjugation of dye molecule to the epitope of the protein).

In another series of embodiments, the present invention provides methods for identifying proteins and other compounds which bind to, or otherwise directly interact with a Class IIa HDAC protein. Thus, in one series of embodiments, High Throughput Screening-derived proteins, DNA chip arrays, cell lysates or tissue homogenates may be screened for proteins or other compounds which bind to one of the normal or mutant Class IIa HDAC genes. Alternatively, any of a variety of exogenous compounds, both naturally occurring and/or synthetic (e.g., libraries of small molecules or peptides), may be screened for Class IIa HDAC function modulating capacity.

Embodiments of the invention also include methods of identifying proteins, small molecules and other compounds capable of modulating the activity of a Class IIa HDAC gene or protein. Using normal cells or animals, the transformed cells and animal models of the present invention, or cells obtained from subjects bearing normal or mutant Class IIa HDAC genes, the present invention provides methods of identifying such compounds on the basis of their ability to affect the expression of a Class IIa HDAC, the activity of a Class IIa HDAC, the activity of proteins that interact with normal or mutant Class IIa HDAC proteins, or other biochemical, histological, or physiological markers that distinguish cells bearing normal and modulated Class IIa HDAC activity in animals.

In accordance with another aspect of the invention, the proteins of the invention can be used as starting points for rational chemical design to provide ligands or other types of small chemical molecules. Alternatively, small molecules or other compounds identified by the above-described screening assays may serve as “lead compounds” in design of modulators of Class IIa HDAC-related traits in animals.

DNA sequences encoding a Class IIa HDAC protein can be expressed in vitro by DNA transfer into a suitable host cell. “Host cells” are cells in which a vector can be propagated and its DNA expressed. The term also includes any progeny or graft material, for example, of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

The terms “recombinant expression vector” or “expression vector” refer to a plasmid, virus or other vehicle known in the art that has been manipulated by insertion or incorporation of a genetic sequence. Such expression vectors contain a promoter sequence which facilitates the efficient transcription of the inserted sequence. The expression vector typically contains an origin of replication, a promoter, as well as specific genes that allow phenotypic selection of the transformed cells.

Methods that are well known to those ordinarily skilled in the art can be used to construct expression vectors containing a Class IIa HDAC coding sequence and appropriate transcriptional/translational control signals. These methods include in vitro recombinant DNA techniques, synthetic techniques, and in vivo recombination/genetic techniques.

A variety of host-expression vector systems may be utilized to express a coding sequence. These include but are not limited to microorganisms such as bacteria transformed with recombinant bacteriophage DNA, plasmid DNA or cosmid DNA expression vectors containing a coding sequence; yeast transformed with recombinant yeast expression vectors containing a coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a coding sequence; insect cell systems infected with recombinant virus expression vectors (e.g., baculovirus) containing a coding sequence; or animal cell systems infected with recombinant virus expression vectors (e.g., retroviruses, adenovirus, vaccinia virus) containing a coding sequence, or transformed animal cell systems engineered for stable expression.

Depending on the host/vector system utilized, any of a number of suitable transcription and translation elements, including constitutive and inducible promoters, transcription enhancer elements, transcription terminators, etc. may be used in the expression vector (see e.g., Bitter et al. Methods in Enzymology 153, 516-544, 1987). For example, when cloning in bacterial systems, inducible promoters such as pL of bacteriophage 7, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used.

The term “operably linked” refers to functional linkage between a promoter sequence and a nucleic acid sequence regulated by the promoter. The operably linked promoter controls the expression of the nucleic acid sequence.

The expression of structural genes may be driven by a number of promoters. Although the endogenous, or native promoter of a structural gene of interest may be utilized for transcriptional regulation of the gene, preferably, the promoter is a foreign regulatory sequence. For mammalian expression vectors, promoters capable of directing expression of the nucleic acid preferentially in a particular cell type may be used (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

Promoters useful in the invention include both natural constitutive and inducible promoters as well as engineered promoters. Examples of inducible promoters useful in animals include those induced by chemical means, such as the yeast metallothionein promoter, which is activated by copper ions (Mett, et al. Proc. Natl. Acad. Sci., U.S.A. 90, 4567, 1993); and the GRE regulatory sequences which are induced by glucocorticoids (Schena, et al. Proc. Natl. Acad. Sci., U.S.A. 88, 10421, 1991). Other promoters, both constitutive and inducible will be known to those of ordinary skill in the art.

Animals included in the invention are any animals amenable to transformation techniques, including vertebrate and non-vertebrate animals and mammals. Examples of mammals include, but are not limited to, pigs, cows, sheep, horses, cats, dogs, chickens, or turkeys.

Compounds tested as modulators of Class IIa HDAC activity can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, modulators can be genetically altered versions of a cellular modulator of Class IIa HDAC activity. Test compounds may be, without limitation, small organic molecules, nucleic acids, peptides, lipids, and/or lipid analogs.

Essentially any chemical compound can be used as a potential modulator or ligand in the assays of the invention, although most often compounds can be dissolved in aqueous or organic solutions. In certain embodiments, the assays of the invention are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of ordinary skill in the art. Such combinatorial chemical libraries include, but are not limited to, peptide libraries (see, e.g., U.S. Pat. No. 5,010,175, Furka, Int. J. Pept. Prot. Res. 37: 487-493, 1991 and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al, Nature Biotechnology, 14: 309-314, 1996 and PCT/US96/10287), carbohydrate libraries (sec, e.g., Liang et al., Science 274: 1520-1522, 1996 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, R U, 3D Pharmaceuticals, Exton, Pa., Martek Biosciences, Columbia, Md., etc.).

Candidate compounds are useful as part of a strategy to identify drugs for inhibiting glucose production wherein the compounds inhibit activity of a Class IIa HDAC, for example, wherein the compound inhibits the binding of HDAC4 and/or HDAC5 or a homolog thereof to one or more interacting proteins, such as HDAC3. Screening assays for identifying candidate or test compounds that bind to one or more cellular modulators of Class IIa HDAC activity, or polypeptides or biologically active portions thereof, are also included in the invention. The test compounds can be obtained using any of the numerous approaches in combinatorial library methods known in the art, including, but not limited to, biological libraries; spatially addressable parallel solid phase or solution phase libraries; synthetic library methods requiring deconvolution; the “one-bead one-compound” library method; and synthetic library methods using affinity chromatography selection. The biological library approach can be used for, e.g., peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and Gallop et al., J. Med. Chem. 37: 1233, 1994.

Libraries of compounds can be presented in solution (e.g., Houghten, Bio/Techniques 13: 412-421, 1992), or on beads (Lam, Nature 354: 82-84, 1991), chips (Fodor, Nature 364: 555-556, 1993), bacteria (U.S. Pat. No. 5,223,409), spores (U.S. Pat. Nos. 5,571,698, 5,403,484, and 5,223,409), plasmids (Cull et al., Proc. Natl. Acad. Sci. USA 89: 1865-1869, 1992) or on phage (Scott et al., Science 249: 386-390, 1990; Devlin, Science 249: 404-406, 1990; Cwirla et al., Proc. Natl. Acad. Sci. USA 87: 6378-6382, 1990; and Felici, J. Mol. Biol. 222: 301-310, 1991).

This invention further pertains to novel agents identified by the herein-described screening assays and uses thereof for treatments as described herein, for example, for the treatment of hyperglycemia in an animal, including humans.

In one embodiment the invention provides soluble assays using an inhibitor of a Class IIa HDAC activity, or a cell or tissue expressing a cellular inhibitor of a Class IIa HDAC activity, either naturally occurring or recombinant. In another embodiment, the invention provides solid phase based in vitro assays in a high throughput format, where a cellular inhibitor of a Class IIa HDAC activity is attached to a solid phase substrate via covalent or non-covalent interactions.

“Inhibitors,” “activators,” and “modulators” of a Class IIa HDAC activity in cells encompass inhibitory, activating, or modulating molecules, respectively, identified using in vitro and in vivo assays for Class IIa HDAC activity, e.g., ligands, agonists, antagonists, and their homologs and mimetics.

“Activity” with respect to a protein includes any activity of the protein, including binding and/or enzymatic activity of the protein.

“Modulator” includes inhibitors and activators. Inhibitors are agents that, e.g., bind to, partially or totally block stimulation, decrease, prevent, delay activation, inactivate, desensitize, or down regulate Class IIa HDAC activity, e.g., antagonists. Activators are agents that, e.g., bind to, stimulate, increase, open, activate, facilitate, enhance activation, sensitize or up regulate a Class IIa HDAC activity, e.g., agonists. Modulators include genetically modified versions of biological molecules with a Class IIa HDAC activity, e.g., with altered activity, as well as naturally occurring and synthetic ligands, antagonists, agonists, small chemical molecules and the like.

“Cell-based assays” for inhibitors and activators include, e.g., applying putative modulator compounds to a biological sample having a Class IIa HDAC activity and then determining the functional effects on the Class IIa HDAC activity, as described herein. “Cell based assays” include, but are not limited to, in vivo tissue or cell samples from a mammalian subject or in vitro cell-based assays comprising a biological sample having Class IIa HDAC activity that are treated with a potential activator, inhibitor, or modulator and are compared to control samples without the inhibitor, activator, or modulator to examine the extent of inhibition.

“Compound” or “test compound” refers to any compound tested as a modulator of Class IIa HDAC activity. The test compound can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, a test compound can be modulators of biological activities that affect a Class IIa HDAC activity. Test compounds may be, without limitation, small organic molecules, nucleic acids, peptides, lipids, and/or lipid analogs.

Methods of delivery of a compound for treatment of a metabolic disease according to the invention include but are not limited to, oral, intra-arterial, intramuscular, intravenous, intranasal, and inhalation routes. In certain embodiments, the delivery route is oral. Suitable modes of delivery will be apparent based upon the particular combination of drugs employed and their known administration forms. A compound for treatment of a metabolic disorder may be administered by any suitable route, including without limitation, oral, rectal, nasal, topical (including transdermal, aerosol, buccal and sublingual), vaginal, penile, parenteral (including subcutaneous, intramuscular, intravenous and intradermal) and pulmonary.

Therapeutic amounts can be empirically determined and may vary with the particular metabolic condition being treated, the subject, the particular formulation components, dosage form, and the like. The actual dose to be administered may vary depending upon the age, weight, and general condition of the subject as well as the severity of the metabolic condition being treated, along with the judgment of the health care professional. Therapeutically effective amounts can be determined by those ordinarily skilled in the art, and will be adjusted to the requirements of each particular case.

The invention will now be described by way of the following non-limiting examples.

### Example 1

### Class IIa HDAC Phosphorylation in Liver is Controlled by LKB1-Dependent Kinases

We sought to identify novel substrates of AMPK and its related family members that mediate control of glucose and lipid metabolism in liver. In a previously described bioinformatics and proteomic screen for substrates of AMPK family kinases (Gwinn et al., 2008; Egan et al., 2011), we identified multiple candidate phosphorylation sites in the Class IIa HDAC family that are highly conserved (FIG. 1A) and represent well-established phosphorylation sites governing their subcellular localization (Haberland et al., 2009). Of the four Class IIa family members in mammals, we examined the protein expression of HDAC4, HDAC5, and HDAC7 in different cell types and used RNAi to validate the specificity of antibodies used for detecting endogenous proteins. HDAC4, HDAC5, and HDAC7 were widely expressed and present in C2C12 myoblasts, embryonic fibroblasts, and hepa1-6 liver-derived cells (FIG. 8A). In order to explore the function and regulation of the Class IIa HDACs in liver, we generated adenoviruses bearing hairpin shRNAs against murine HDAC4, HDAC5, and HDAC7, which efficiently knocked down each family member (FIG. 1B). As each family member was up-regulated when another was depleted (FIG. 1B), to study loss of Class IIa HDAC function it was important to combine shRNAs of all three.

Phospho-specific antibodies were validated for detecting endogenously phosphorylated HDAC4, HDAC5, and HDAC7 on their Ser259 and Ser 498 sites (FIG. 1B, 8B, supplementary text), and used to examine whether these sites in each family member were regulated by LKB1-dependent kinases in liver or hepatoma cell lines. Consistent with previous reports suggesting AMPK family members can target Class IIa HDACs in other cell types (Berdeaux et al., 2007; Dequiedt et al., 2006; McGee et al., 2008; Van der Linden, 2006), RNAi depletion of LKB1 resulted in loss of basal Phospho-Ser259 and Phospho-Ser498 of HDAC4 and HDAC5 in HepG2 and Huh7 hepatoma cells (FIG. 1C). Moreover, treatment with phenformin, which activates AMPK in an LKB1-dependent manner, also led to an LKB1-dependent increase in phosphorylation on Ser498 of HDACs4/5 (FIG. 1C, 8C,1D,1E, supplemental text).

To examine the physiological conditions when Class IIa HDACs are regulated by the LKB1 pathway, we utilized a conditional deletion of the LKB1 gene in mouse liver (Shaw et al., 2005). LKB1 deletion led to loss of basal Phospho-Ser259 and Phospho-Ser498 in HDACs4/5/7, and acute treatment of mice with the AMPK agonist metformin led to an increase in Phospho-Ser498 in HDAC4/5/7 (FIG. 1D), consistent with results from hepatoma cell lines. Paralleling the effects seen with metformin and phenformin, A769662, a direct AMPK activating small molecule (Cool et al., 2006), increased HDAC4/5/7 phosphorylation, particularly on the Ser498 sites (FIG. 8F). Collectively, these data indicate Class IIa HDACs are bona fide in vivo targets suppressed by the LKB1 signaling pathway in liver, and can be further inhibited in response to the anti-diabetic compound metformin.

### Example 2

### The Fasting Hormone Glucagon Induces Dephosphorylation and Nuclear Shuttling of Class IIa HDACs

Considering the prominent basal phosphorylation of the HDACs in primary hepatocytes and in livers of ad lib fed mice (FIG. 1B,D), we sought to examine whether their phosphorylation may be controlled by physiological stimuli such as fasting and re-feeding and discovered that HDAC4/5/7 phosphorylation in the liver was reduced under fasting conditions and increased upon re-feeding (FIG. 2A). To examine whether this was an adaptive response to fasting, or whether hormones induced upon fasting could acutely mimic this effect, mice were injected with the fasting hormone glucagon, which resulted in reduced HDAC4/5/7 phosphorylation (FIG. 9A). The observed decrease of HDAC4/5/7 phosphorylation by glucagon paralleled decreased phosphorylation of CRTC2, another protein whose localization is controlled by LKB1-dependent kinases and 14-3-3 binding (Screaton et al., 2004). To further define the effects of glucagon, we examined the phosphorylation and localization of the HDACs in primary hepatocyte cultures. Consistent with the high basal levels of endogenous HDAC4/5/7 phosphorylation observed in primary hepatocytes, GFP tagged-HDAC5 was basally excluded from the nucleus of these cells (FIG. 2D). Treatment with glucagon induced rapid loss of endogenous HDAC4/5/7 phosphorylation (FIG. 2B) and full nuclear translocation of GFP-tagged HDAC5 within 30 minutes (FIG. 2D). Similar results were observed with forskolin, another cAMP-inducing compound (FIG. 9B,C). No such effect was observed for GFP alone or the non-phosphorylatable Ser259Ala, Ser498Ala (AA) GFP-HDAC5 mutant, which exhibited a permanent nuclear localization identical to wild-type HDAC5 localization following glucagon or FSK treatment (FIG. 9D). Subcellular fractionation corroborated that under basal conditions in primary hepatocytes, endogenous Class IIa HDACs are predominantly cytoplasmic and translocate fully into the nucleus following glucagon or forskolin treatment (FIG. 2C).

### Example 3

### Class IIa HDACs are Required for Expression of Glucagon-Induced Gluconeogenic Genes

These findings indicate that Class IIa HDACs in liver may be acting as fasting-induced modulators of transcription. Knowing that glucagon induced their nuclear translocation, we hypothesized that their direct involvement in control of transcription should occur acutely following hormone treatment. We therefore performed transcriptional profiling analysis in primary hepatocytes to define the genes whose expression is altered by forskolin in a manner that is suppressed by HDAC4/5 shRNAs. Contrary to our initial expectations that the Class IIa HDACs would act as fasting-induced transcriptional repressors, amongst the genes regulated by forskolin, we observed far more genes whose expression was attenuated when HDAC4/5 were depleted via shRNA (heatmap of 15 representative genes selected from the top 50 HDAC4/5 regulated genes in FIG. 3A; top 25 HDAC4/5 regulated genes shown in FIG. 10A; full dataset GEO submission GSE20979).

Strikingly, the single most-regulated gene on the entire array following knockdown of Class IIa HDACs was the catalytic subunit of G6Pase (G6pc), a rate-limiting enzyme for gluconeogenesis and glycogenolysis (FIG. 10A). In addition to G6Pase, forskolin-induced expression of the other rate-limiting gluconeogenic genes PEPCK (Pck1) and Fbp1 was similarly attenuated when HDAC4/5 were depleted. Several of the HDAC4/5-regulated genes from the array are known to be FOXO and/or CREB target genes, and we further validated their HDAC-regulation by Q-PCR (FIG. 3B). We next examined whether the effect of HDAC4/5/7 on transcription of these loci could be observed on a reporter consisting of 2.2 KB of the human G6Pase promoter driving luciferase expression. Similar to the effect on endogenous G6Pase mRNA expression, shRNA-mediated depletion of HDAC4/5/7 inhibited the induction of luciferase from the G6Pase promoter following forskolin treatment in hepatocytes (FIG. 3C, top panel), comparable to loss of CRTC2 expression, which is needed for CREB-dependent transactivation of the G6Pase promoter. In addition, over-expression of constitutively nuclear non-phosphorylatable S259A/S498A HDAC5 mutant resulted in a modest but reproducible increase in basal G6Pase reporter activity even in the absence of forskolin, and further potentiated the effect of forskolin mediated induction. In contrast, HDAC4/5/7 depletion did not alter forskolin-induction of a CRE-luciferase reporter composed of 3 tandem copies of the CREB DNA binding consensus motif compared to the effect of CRTC2 shRNA (FIG. 3C, bottom panel). Consistent with the results in hepatocytes, depletion of HDAC4/5/7 in vivo resulted in attenuation of G6Pase promoter activity, but had no effect on the CRE-luciferase reporter in murine liver (FIG. 3D, data not shown). No significant changes in protein levels of CREB, PGC-1a, CRTC2, or of the two FOXO family members expressed in the liver, Foxo1 or Foxo3 were seen with HDAC4/5/7 knockdown (FIG. 10B).

Given the effects on the G6Pase reporter, we examined next whether endogenous HDAC4 or HDAC5 may be recruited to the G6Pase promoter following glucagon treatment using chromatin immunoprepitation (ChIP). As seen in FIG. 3E, endogenous HDAC4 and HDAC5 were immunoprecipitated in a glucagon-inducible manner with a proximal promoter region of the G6Pase promoter containing the FOXO and CREB consensus binding sites (Vander Kooi et al., 2003). In the absence of glucagon, no association of HDAC4 or HDAC5 was observed with this region above background, or with a non-specific distal upstream or internal regions (FIG. 3E; FIG. 10C). shRNA confirmed the specificity of the ChIP signal at the G6Pase and Pck1 loci (FIG. 10D).

### Example 4

### Class IIa HDACs Control Acetylation of FOXO Transcription Factors Via Class I HDAC3

Given the association of HDAC4 and HDAC5 with the G6Pase promoter following glucagon, we investigated whether the presence of Class IIa HDACs may be modulating the acetylation of one of the transcription factors or transcriptional co-activators required for G6Pase induction following glucagon. To further investigate whether Class IIa HDACs may be affecting the acetylation of these transcription factors, we tested whether they physically associate. We found significant co-immunoprecipitation of FOXO1 or FOXO3 with HDAC5 following forskolin treatment (FIG. 4A, 11A, data not shown). Consistent with this interaction, we observed both endogenous Foxo1 and endogenous HDAC4 to be nuclear following forskolin treatment of primary hepatocytes (FIG. 4B).

Foxo1 is acetylated on Lys242, 245, 259, 262, 271, and 291 by the histone acetyltransferases p300 and CBP, which reduces its ability to bind DNA (Brent et al., 2008; Matsuzaki et al., 2005). Using acetylation site-specific antibodies, we examined FOXO1 or FOXO3 acetylation in primary hepatocytes treated with shRNAs against the Class IIa HDACs. Acetylation of Foxo1 and Foxo3 was dramatically increased as measured using anti-Acetyl Lys259/262/271 Foxo1 antibody (FIG. 4C,D), while histone3 Lys9/Lys14 acetylation remained unchanged. Identical results were observed with an acetylation specific antibody to the nearby Lys242/245 sites in Foxo1 (Matsuzaki et al., 2005) (FIG. 11C). Importantly, adenoviral mediated knockdown of HDAC4/5/7 in mouse liver led to increased acetylation of endogenous FOXO1 (FIG. 4E). Acetylation of FOXO has been reported to reduce its DNA binding, making it more accessible for Akt and related inactivating kinases (Jing et al., 2007; Qiang et al., 2010). Consistent with the increase of acetylation, knockdown of HDAC4/5/7 in hepatocytes led to an increase of Akt-dependent phosphorylation of endogenous Foxo1 and Foxo3 (FIG. 4F).

As previous studies indicate that Foxo1 acetylation on Lys242/245 directly disrupts its ability to bind DNA (Matsuzaki et al., 2005; Brent et al., 2008), we examined the association of Foxo1 with gluconeogenic promoters. Glucagon treatment resulted in increased ChIP of endogenous Foxo1 with the G6Pase and PEPCK promoters, which was attenuated by HDAC4/5/7 shRNA, consistent with increased FOXO acetylation and loss of DNA binding (FIG. 5A).

Several studies have suggested the Class IIa HDACs are catalytically inactive due to critical amino acid substitutions within the catalytic residues (Lahm et al., 2007; Schuetz et al., 2008). In other contexts where Class IIa-associated deacetylase activity was detected, it was attributed to Class IIa HDACs association and recruitment of active Class I HDAC family member HDAC3 and its co-regulators Ncor1/SMRT(Ncor2) (Fischle et al., 2002). Consistent with this possibility, we observed that overexpressed HDAC5 and HDAC3 co-immunoprecipitated in a forskolin-dependent manner in HEK293 cells and endogenous HDAC3 and Foxo1 co-immunoprecipitated with GFP-HDAC5 from hepatocytes in a glucagon-dependent manner (FIG. 5B, 12A). Moreover, we found that recombinant HDAC4 or 5 were unable to stimulate in vitro deacetylation of FOXO1, unlike recombinant HDAC3/Ncor complex (FIG. 5C, FIG. 12B). The ability of HDAC3 to catalyze in vitro deacetylation of FOXO was dependent on its association with Ncor (FIG. 5C), as previously reported in other deacetylase assays (Fischle et al., 2002; Gregoire et al., 2007). Consistent with these findings, treatment of cells with the Class I/II HDAC inhibitor trichostatin A (TSA) results in increased FOXO1 acetylation (FIG. 12C), as reported previously (Brunet et al. 2004).

To further examine if HDAC3 may mediate FOXO deacetylation in concert with HDAC4/5 in hepatocytes, we looked at whether HDAC3 similarly associated with the same regulatory regions of the G6Pase and PEPCK promoters, and whether this association was regulated by glucagon. ChIP experiments revealed that endogenous HDAC3 bound to both the G6Pase and PEPCK promoters only following glucagon treatment, and this association was abolished when HDAC4/5/7 were depleted (FIG. 5D), in contrast to its association with the promoter of the housekeeping gene TFIIB. Taken altogether, these findings substantiate the model that following glucagon, Class IIa HDACs translocate into the nucleus where they recruit HDAC3 to the G6Pase and PEPCK promoters. HDAC3 contains deacetylase activity towards FOXO, promoting its activation and induction of these gluconeogenic gene promoters.

### Example 5

### Suppression of Class IIa HDACs Alters Organismal Glucose Homeostasis

G6Pase is a rate-limiting enzyme of both gluconeogenesis and glycogenolysis (Hutton and O'Brien, 2009) and mutations in glucose-6-phophatase (G6pc) result in Glycogen Storage Disease Type I in humans (GSD Type I or Von Gierke's disease) characterized by aberrant glycogen storage and hypoglycemia, a phenotype also mimicked in genetic mouse models of G6Pase deletion (Salganik et al., 2009; Peng et al., 2009). Given the dramatic effect of HDAC4/5/7 depletion on G6Pase in hepatocytes, we sought to examine the effect of their loss in the intact mouse liver. Similar to mice lacking G6Pase or Foxo1 (Matsumoto et al., 2007), mice expressing shRNAs against HDAC4 or HDAC5 alone in liver give rise to increased glycogen accumulation as visualized by Peroidic acid-Schiff (PAS) stain in both fasting and refed mice (FIG. 6A). The most significant effect on glycogen accumulation was observed when HDAC4, HDAC5, and HDAC7 were all simultaneously knocked down (FIG. 6A; quantified in FIG. 13A). We also observed that loss of HDAC4/5/7 modestly lowered blood glucose levels in B6 mice on a normal diet, and importantly, over-expression of non-phosphorylatable constitutively nuclear HDAC5 led to a modest increase in blood glucose in these mice (FIG. 13B). B6 mice expressing hepatic HDAC4/5/7 shRNA also showed improved glucose tolerance in a glucose tolerance test (FIG. 13C). Gain and loss of Class IIa HDAC function in fasted B6 mice correlated with changes in G6Pase mRNA levels (FIG. 6B), similar to the effects observed on the G6Pase reporter in hepatocytes (upper panel of FIG. 3C).

As hepatic deletion of LKB1 leads to the loss of HDAC4/5/7 phosphorylation (FIG. 1D), HDAC4/5/7 will be constitutively nuclear in LKB1−/− livers, potentially contributing to increased gluconeogenic gene expression. To examine whether constitutive activation of HDAC4/5/7 may play a role in the hyperglycemia of hepatic LKB1 knockout mice, we combined a model of inducible loss of hepatic LKB1 in mice with subsequent introduction of adenoviral shRNA against HDAC4/5/7. We utilized liver-specific inducible Cre recombinase transgenic mice (Imai et al., 2000) crossed to the LKB1 conditional floxed knockout mice. Consistent with previous results of Cre mediated LKB1 loss, tamoxifen induced loss of hepatic LKB1 led to a doubling of fasting blood glucose levels within 10 days post administration. Subsequent loss of HDAC4/5/7 in these mice led to remarkable suppression of the LKB1-dependent elevation in blood glucose (FIG. 6C). Immunoblotting confirmed that LKB1 and HDAC4/5 expression were attenuated and that in the absence of LKB1 expression in liver, HDAC4 and 5 were basally hypo-phosphorylated (FIG. 6E). We next looked at the expression levels of FOXO regulated genes in the context of Class IIa HDAC loss in this mouse model. Indeed, in addition to G6Pase and PEPCK, the expression of several FOXO target genes was significantly elevated in the LKB1−/− livers compared to LKB1+/+ livers, and were subsequently reduced following HDAC4/5/7 depletion in those livers and not in control scrambled shRNA expressing livers (FIG. 6D, data not shown).

### Example 6

### Class IIa HDACs are Required for Hyperglycemia in Diabetic Mouse Models

Given that insulin resistance associated with the metabolic syndrome is known to result in FOXO-dependent increases in gluconeogenesis (Gross et al., 2009), we sought to more broadly examine whether deregulation of HDAC4/5/7 function may contribute to hyperglycemia in widely used mouse models of type 2 diabetes and whether targeting their inactivation would be sufficient to restore glucose homeostasis in this setting. First, we utilized the ob/ob and db/db mouse models deficient in leptin signaling and deregulated for insulin signaling, and treated these mice with either scrambled control or HDAC4/5/7 shRNAs as above. Reduction of Class IIa HDAC expression in these diabetic mouse models also led to a substantial decrease in fasting blood glucose levels, (FIG. 7B, 14A), paralleling loss of HDAC expression (FIG. 14B). To more fully characterize this response, we performed glucose tolerance tests (GTTs) and pyruvate tolerance tests (PTTs) on db/db cohorts treated with control or HDAC4/5/7 shRNAs. Loss of the Class IIa HDACs significantly lowered fasting blood glucose levels and improved glucose tolerance in db/db mice (FIG. 7A,C). Next we examined whether the Class IIa HDACs were also involved in regulating hepatic blood glucose in a high fat diet induced diabetes mouse model, which is thought to be more representative of human type 2 diabetes onset. The high fat diet (HFD) mice also showed a significant reduction of fasting blood levels and improved glucose tolerance when depleted for HDAC4/5/7 in the liver (FIG. 7D,E), indicating that the Class IIa HDACs play a critical role in controlling hepatic glucose homeostasis.

### Example 7

### Experimental Procedures

Antibodies and Biochemistry

Cell Signaling antibodies used: pAMPK, pACC, pRaptor, Raptor, HDAC3, HDAC4, HDAC5, pHDAC4 Ser246/HDAC5 Ser259/HDAC7 Ser155, pHDAC4 Ser632/HDAC5 Ser498/HDAC7 Ser486, SIRT1, LKB1, Foxo1, Foxo3, pFoxo, CREB, Myc, GST. Millipore antibodies used: LKB1, Histone3 K9/K14, Acetyl Lysine. Santa Cruz antibodies used: Ac-Foxo1, αTubulin, HDAC7. Abcam antibodies used: HDAC3. Sigma antibodies used: M2 Flag, anti-Flag. Anti-CRTC2 and PGC1a previously described (Dentin et al., 2009). All catalog numbers and buffers described in Extended Experimental Procedures.

DNA Constructs and Adenoviruses

GST-14-3-3, Myc CA-AMPKα2, GFP HDAC5 WT, GFP HDAC5 S259A/S498A, and Myc-Foxo1 described previously (Gwinn et al., 2008; Berdeaux et al., 2007). FLAG HDAC5 WT, Flag tagged WT HDAC3, GFP Foxo1 Myc Foxo1 obtained from Addgene. FLAG HDAC5 S259A and FL HDAC5 S259A/S498A generated using QuickChange Site-Directed Mutagenesis kit (Stratagene). For full details on adenoviruses used and adenoviral construction see Extended Experimental Procedures.

Cell Culture

HEK293T, Huh7, HepG2, C2C12, and U2OS cells were obtained from ATCC. RNAi SMARTpool human LKB1 (Dharmacon) or RNAi negative control (Invitrogen) used at 20 nM final concentration and transfected using RNAiMAX transfection reagent (Invitrogen). Knockdowns were carried out for 72 hrs. Cells were treated with 1 uM TSA or 10 mM NAM (Sigma), Cells were treated with 2 mM AICAR (Toronto Research Chemicals) or 2 mM Phenformin (Sigma).

Primary Hepatocyte Treatment and Subcellular Fractionation

Primary hepatocytes were derived from C57BL/6J mice and maintained in serum free Media 199. Cells were transduced 24 hrs after harvesting. Knock-down and over-expression studies in hepatocytes were done by infecting cells at 5 PFUs/cell. All adenoviral shRNA knockdowns were carried out for 72 hrs. For subcellular fractionation, cells were treated as indicated, washed 3 times with PBS and lysed utilizing NE-PER Cell Fractionation Kit (Pierce). Primary hepatocytes were treated with 10 uM Forksolin (Sigma) and 100 nM Glucagon (Novo Nordisk), 100 nM insulin (Lilly) at indicated times.

Chromatin Immunoprecipitation

Primary hepatocytes were stimulated with PBS or 100 nM glucagon and fixed in 1% formaldehyde. Nuclear extracts were sonicated and precleared with normal rabbit IgG (Santa Cruz Biotechnology). Chromatin was immunoprecipitated with anti-HDAC4 (CST, #2072), anti-HDAC5 (CST, #2082), anti-HDAC3 (Abcam), anti-Foxo1 (A. Brunet) or normal rabbit IgG. Immunoprecipitated chromatin was decrosslinked, ethanol precipitated and quantified by SYBR green quantitative PCR. Recoveries were calculated as percent of input.

Animal Experiments and Procedures

LKB1lox/lox mice (Shaw et al., 2005) were crossed to Albumin-creERT2 mice (Imai et al., 2000). To induce Cre-mediated deletion in Albumin-creERT2 mice, mice were intraperitoneally injected with 1 mg/mouse of Tamoxifen (SIGMA) for 5 consecutive days. Ad-Cre mediated deletion in LBK1lox/lox mice was done by tail vein injection of 1×109 PFUs/mouse in 8 week old males (FIG. 1D). C57BL/6J, db/db, ob/ob, and C57BL/6J High fat diet-fed mice (60% kcal %, Research Diets Incorporated D12492i) obtained from Jackson Laboratories. For metformin experiments, mice injected intraperitoneally with 250 mg/kg Metformin in 0.9% saline for 1 h. For basal blood glucose, mice were fasted 18 h o/n and then glucose was measured using a glucometer (Bayer). All animal care and treatments were in accordance with the Salk Institute guidelines for the care and use of animals (IACUC protocol 08-045). For additional details see Extended Experimental Procedures.

qPCR Analysis

mRNA from primary hepatocytes was isolated using RNAeasy (Qiagen) kit and reverse transcribed using SuperScript II Reverse Transcriptase. Three samples/mice were used per condition and qPCR was done in technical triplicate for each sample. qPCR reaction was carried out using Syber GreenER (Invitrogen). All qPCR results are representative of 3 separate experiments.

Statistical Analysis

Comparisons were made using the unpaired Student's t-test. SEM+/− is represented as error bars. Statistical significance as indicated.

### Example 8

### Class IIa HDACs Antisera and their Subcellular Localization

To rigorously validate the endogenous proteins detected by immunoblotting, we generated adenoviruses bearing hairpin shRNAs directed against HDAC4, HDAC5, and HDAC7. Similar to Hepa1-6 cells, in cultured primary hepatocytes, as well as in intact livers harvested from mice tail-vein injected with adenoviral shRNAs, endogenous HDAC4 and HDAC5 proteins were readily detected (FIG. 1B). Notably, shRNA against HDAC4 or HDAC7 resulted in up-regulation of HDAC5 protein levels, whereas shRNA against HDAC5 or HDAC7 resulted in up-regulation of HDAC4 protein levels, indicating significant compensatory regulatory mechanisms at the protein level. Thus, to study loss of ClassIIa HDAC function in hepatocytes, it is important to combine shRNAs to HDAC4, HDAC5, and HDAC7 (FIG. 1B). This compensatory upregulation of family members was not observed in the cell lines in FIG. 8A.

To examine whether the well-established regulatory phosphorylation sites conserved in Class II HDACs were regulated in these cells, we characterized phospho-specific antibodies against the two best studied sites: Serine259 in HDAC5 (Ser246 in HDAC4/Ser155 HDAC7) and Serine498 in HDAC5 (Ser467 in HDAC4/Ser358 in HDAC7). The importance of these sites in the regulation of 14-3-3 binding and nuclear/cytoplasmic shuttling of the Class IIa HDACs has been well-established in previous studies (Grozinger and Schreiber, 2000; McKinsey et al., 2000a,b; Wang et al., 2000; Zhao et al., 2001; Vega et al., 2004).

Importantly, the flanking residues recognized by the Phospho-Ser259 antibody are completely conserved between HDAC4 and HDAC5, and differ only by a single subtle residue substitution in HDAC7, allowing these antibodies to detect all three of these Class IIa HDAC family members (FIG. 1A,B). Full-length HDAC4 and HDAC5 are similar in molecular weight and migrate at the same position of SDS-PAGE (˜140 kD), whereas HDAC7 in liver primarily migrates at 105 kD (FIG. 1B). After verifying the phospho-specificity of the antibodies (FIG. 8B), an examination of endogenous HDAC proteins in liver lysates revealed that HDAC4 shRNA resulted in a loss of ˜50% of the 140 kD band recognized by the Phospho-Ser259 antibody, and similarly HDAC5 shRNA reduced the other 50% of this band. shRNA to both HDAC4 and HDAC5 resulted in a near complete loss of this band, but not of the 105 kD band recognized by the antibody which was abolished by HDAC7 shRNA (FIG. 1B). These RNAi experiments indicate that endogenous hepatic HDAC4, HDAC5, and HDAC7 are all expressed and recognized by the Ser259 and Ser498 antibodies we are utilizing. It also indicates that the phosphorylation of these three family members is coordinately regulated in murine liver and the cell lines examined.

Importantly, whether AMPK activating compounds alter the subcellular localization of the Class IIa HDACs depends on the basal phosphorylation state of these proteins in the cell line and culture conditions being examined. In growing U2OS osteosarcoma tumor cells, the Class IIa HDACs are not significant basally phosphorylated and are nuclear when overexpressed, hence shuttle in response to AMPK activators (FIG. 8C). Importantly, their 14-3-3 association and cytoplasmic shuttling occurs when co-expressed with constitutively active AMPK and is not seen with non-phosphorylatable serine-to-alanine HDAC5 mutants (FIG. 8D, 8E). In contrast to U20S cells, in primary hepatocytes and fed murine liver, Class IIa HDACs are fairly highly phosphorylated in the basal state hence the degree of phosphorylation or cytoplasmic shuttling induced by AMPK activation is less than observed in the U2OS cells. Collectively, we expect the localization of Class IIa HDACs and its reliance on AMPK or other LKB1-dependent kinases will rely on the cell line and mostly reflect the expression and activity of the various LKB1-dependent kinases as well as other kinases that can also phosphorylate these sites, including PKD and members of the CAMK family.

### Example 9

### Additional Experimental Procedures

Antibodies and Biochemistry

Except where noted, cell and liver extracts were prepared in 20 mM Tris pH 7.5, 150 mM NaCl, 1 mM EDTA, 1 mM EGTA, 1% Triton X-100, 2.5 mM pyrophosphate, 50 mM NaF, 5 mM b-glycero-phosphate, 50 nM calyculin A, 1 mM Na3VO4, 10 mM PMSF, 4 mg/ml leupeptin, 4 mg/ml pepstatin, 4 mg/ml aprotinin) lysis buffer. Total protein was normalized using BCA protein kit (Pierce) and lysates were resolved on SDS-PAGE gel.

Cell Signaling Antibodies used: pAMPK Thr172 (#2535), pACC Ser79 (#3661), pRaptor Ser792 (#2083), total Raptor (#2280), total HDAC3 (#3949), total HDAC4 (#2072), total HDAC4 (#5392), total HDAC5, (#2082), pHDAC4 Ser246/HDAC5 Ser259/HDAC7 Ser155 (#3443), phospho-HDAC4 Ser632/HDAC5 Ser498/HDAC7 Ser486 (#3424), total SIRT1 (#2028), LKB1 (#3050), Foxo1 (#2880), Foxo3 (#9467), Phospho-Foxo (#9464), (CREB (#9197), Myc (#2278), Myc (#2276), GST (#2622). Millipore antibodies used: LKB1 (#07-694), Histone3 K9/K14 (#06-599), Acetyl Lysine (#05-515). Santa Cruz antibodies used: Ac Foxo1 (#sc49437), αTubulin (SC53029), HDAC7 H237 (sc-11421). Abcam antibodies used: HDAC3 (ab7030), HDAC3 (ab11967). Sigma antibodies used: M2 Flag (#F1804), anti-Flag (#F7425). Anti-CRTC2 and PGC1a was as previously described (Dentin et al., 2009).

Primary Hepatocyte Luciferase Assays

Primary hepatocytes were harvested from wild type C57BL/6J mice and cultured in serum free Medium 199 and infected at 5 PFUs/cell with scrambled control shRNA, HDACs shRNAs or CRTC2 shRNA (for 72 h total). After 24 h, cells were co-infected with reporters, CRTC2 WT or HDAC5-AA expressing adenovirus for 40-48 h. Cells were treated with either vehicle or 10 uM Forskolin for 4 hours and lysed in passive lysis buffer (Promega) and analyzed using Dual-Glo Luciferase Reporter System (Promega) Firefly luciferase signal was normalized to Renilla luciferase or B-galactosidase. All infections and treatments were done in triplicates and readouts were then averaged.

Generated and Purchased Adenoviruses

The following adenoviruses were purchased from Vector Biolabs: Ad-U6-Scramble (scram) RNAi-GFP (#1122), Foxo3A (#1026), Ad-pRenilla-Luc (#1671). pAD GFP was purchased from Eton Bioscience (#0100032001). The following adenoviruses were previously described: pAd RSV-Bgal, pAd G6Pase-luc, pAd CRE-luc, pAd Foxo1 shRNA, pAd CRTC2 shRNA, pAd US scrambled RNAi, pAd wild type CRTC2 (Dentin et al., 2007). pAd CMV CRE was purchased from University of Iowa Gene Transfer Vector Core (Iowa City, Iowa). GFP Foxo1 WT adenovirus was provided by D. Accili (Columbia University). GFP HDAC5 WT and GFP HDAC5 S259A/S498A expressing adenoviruses were generated using the pAd/CMV/V5-DEST vector of Gateway Cloning Technology (Invitrogen) starting from previously described GFP-HDAC5 constructs. HDAC specific shRNA expressing adenoviruses were generated using pAd BLOCK-iT Adenoviral RNAi Expression System (Invitrogen). Three separate hairpins per gene were generated and tested for knockdown efficiency in preliminary experiments. All Adenoviruses were generated in large scale preps using forty 15 cm plates of 293 E4 cells, CsCl purified and functional viral titers were obtained using an Elisa assay for the detection of hexon, fiber and penton capsid proteins. The following sequences were used to generate mouse specific shRNAs against the Class IIa HDACs:

mHDAC4:
5′-GGTACAATCTCTCTGCCAAATCGAAATTTGGCAGAGAGATT
GTACC-3′
3′-CCATGTTAGAGAGACGGTTTAGCTTTAAACCGTCTCTCTAA
CATGG-5′
mHDAC5:
5′-GGCTCAGACAGGTGAGAAAGACGAATCTTTCTCACCTGTCT
GAGCC-3′
3′-CCGAGTCTGTCCACTCTTTCTGCTTAGAAAGAGTGGACAGA
CTCGG-5′
mHDAC7:
5′-GGGTCGATACTGACACCATCTCGAAAGATGGTGTCAGTATC
GACCC-3′
3′-CCCAGCTATGACTGTGGTAGAGCTTTCTACCACAGTCATAG
CTGGG-5′

Animal Adenovirus Experiments

shRNA mediated knockdown in mouse livers was done through tail vein injection of mice at 1×109 PFUs/mouse for all pAd shRNAs and GFP-HDAC5-AA mutant, 5×108 PFUs/mouse for G6Pase-luc reporter, and 1×108 PFUs for RSV-Bgal and Ad-pRenilla-Luc reporters. Livers were harvested or imaged 4-6 days following adenoviral delivery.

qPCR Analysis
qPCR Primers used (5′ to 3′):
1. Agxt212
Forward Primer: AGAGGGAGGAACATTCATTGACT
Reverse Primer: GGCTCGCATTATTTTGATGGGA
2. PEPCK
Forward Primer: CTGCATAACGGTCTGGACTTC
Reverse Primer: CAGCAACTGCCCGTACTCC
3. Mmd2
Forward Primer: AGTATGAACACGCAGCAAACT
Reverse Primer: TCCCAGTCGTCATCGGACA
4. IGFBP1
Forward Primer: ATCAGCCCATCCTGTGGAAC
Reverse Primer: TGCAGCTAATCTCTCTAGCAC
5. HDAC4
Forward Primer: CAAGGAGAAGGGCAAAGAGA
Reverse Primer: TCCTGCAGCTTCATCTTCAC
6. G6Pase:
Forward Primer ACTGTGGGCATCAATCTCCTC
Reverse Primer CGGGACAGACAGACGTTCAGC
7. SGK1
Forward Primer: CTGCTCGAAGCACCCTTACC
Reverse Primer: TCCTGAGGATGGGACATTTTCA
8. Cyclophilin:
Forward Primer: TGGAGAGCACCAAGACAGACA
Reverse Primer: TGCCGGAGTCGACAATGAT

Microarrays Analysis

Total RNA was extracted using Trizol reagent (Invitrogen) and purity of the RNA was assessed by Agilent 2100 Bioanalyzer. 500 ng of RNA was reverse transcribed into cRNA and biotin-UTP labeled using the Illumina TotalPrep RNA Amplification Kit (Ambion). cRNA was quantified using an Agilent Bioanalyzer 2100 and hybridized to the Illumina mouseRefseq-8v1.1 Expression BeadChip using standard protocols (Illumina). Image data was converted into unnormalized Sample Probe Profiles using the Illumina BeadStudio software and analyzed on the VAMPIRE microarray analysis framework. Stable variance models were constructed for each of the experimental conditions (n=2). Differentially expressed probes were identified using the unpaired VAMPIRE significance test with a 2-sided, Bonferroni-corrected threshold of αBonf=0.05. The VAMPIRE statistical test is a Bayesian statistical method that computes a model-based estimate of noise at each level of gene expression. This estimate was then used to assess the significance of apparent differences in gene expression between 2 experimental conditions. Lists of altered genes generated by VAMPIRE were mapped to pathways using the VAMPIRE tool GOby to determine whether any KEGG categories were overrepresented using a Bonferroni error threshold of αBonf=0.05. Heat map was constructed using the CIMminer program at http://discover.nci.nih.gov/, a development of the Genomics and Bioinformatics Group, Laboratory of Molecular Pharmacology (LMP), Center for Cancer Research (CCR) National Cancer Institute (NCI).

Immunoflourescence

Cells were washed three times with PBS and fixed in 4% cold PFA. Anti-Myc (9B11, Cell Signaling Technology #2276) ab was used for detecting Myc-AMPKa2. Secondary antibodies were anti-rabbit Alexa488 and anti-mouse Alexa594 (Molecular Probes, 1:1000). DNA was stained with DAPI. Coverslips were mounted in FluoromountG (SouthernBiothech). Images were acquired on a Zeiss Axioplan2 epifluorescence microscope coupled to the Openlab software. Images were acquired using the 63× objective. Immunoflourescence to detect endogenous Foxo1 and HDAC4 was performed on primary hepatocytes treated either with vehicle (DMSO) or 10 uM Forskolin for 1 hr.

Confocal Imaging Analysis Primary hepatocytes were treated either with Vehicle (media) or 100 nM Glucagon for indicated times, washed in PBS and fixed with 4% PFA. Confocal microscopy was performed on an LSM 710 spectral confocal microscope mounted on an inverted Axio Observer Z1 frame (Carl Zeiss, Jena, Germany). Excitation for both markers was provided by a 405 nm solid-state diode laser (for DAPI) and the 488 nm line of an Argon-ion laser (for green) respectively. Laser light was directed to the sample via two separate dichroic beamsplitters (HET 405 and HFT 488) through a Plan-Apochromat 63×1.4 NA oil immersion objective (Carl Zeiss, Jena Germany). Fluorescence was epi-collected and directed to the detectors via a secondary dichroic mirror. DAPI fluorescence was detected via a photomultiplier tube (PMT) using the spectral window 430-480 nm. Green fluorescence was detected on a second photomultipler tube (PMT) with a detection window of 500-570 nm. Confocal slice thickness was typically kept at 0.8 microns consistently for both fluorescence channels with 10 slices typically being taken to encompass the three-dimensional entirety of the cells in the field of view. Maximum intensity projections of each region were calculated for subsequent quantification and analysis.

Acetylation Assessment

In vivo: Primary hepatocytes were isolated from wild type C57BL/6J mice and cultured in serum free Medium 199 (Mediatech, 5.5 mM glucose) after attachment. Cells were infected with adenoviruses encoding control scrambled shRNA and/or HDAC4/5/7 shRNAs for a total of 72 h of knockdown. Cells were transduced with either wild-type Foxo3a or wild-type GFP-Foxo1 expressing adenovirus for 24 h prior to cell lysis in scrambled or HDACs shRNAs infected cells. Lysis buffer (20 mM Tris pH 7.5, 150 mM NaCl, 1 mM EDTA, 1 mM EGTA, 1% Triton X-100, 2.5 mM pyrophosphate, 50 mM NaF, 5 mM β-glycero-phosphate, 50 nM calyculin A, 1 mM Na3VO4) contained 5 uM TSA and 10 mM Nicotinamide. Total protein was normalized by modified BCA protein analysis and resolved on SDS-PAGE gel. Foxo1/3 acetylation and HDAC knockdown was assessed using the above-mentioned antibodies. Endogenous Foxo1 acetylation was assessed in Foxo1 immunoprecipitates from mouse livers expressing scrambled or HDAC4/5/7 shRNAs.

In vitro: The following recombinant proteins were purchased from Enzo Life Sciences: recombinant HDAC 1 (BML-SE456), HDAC3 (BML-SE507), HDAC3/NCoR1 (BML-SE515), SIRT1 (BML-SE239). The following recombinant proteins were purchased from Millipore: p300, HAT domain (14-418), HDAC4 (14-828), Foxo1 (14-343). Recombinant HDAC5 (H87-31G) was purchased from SignalChem. Invitro acetylation reactions were performed by incubating 2 ug of recombinant GST Foxo1 with recombinant HAT fragment of p300 for 1 hour at 30 degrees. Reactions were carried out in acetylation buffer containing 50 mM Tris HCl (pH 8.0), 0.1 mM EDTA, 1 mM DTT, 10% glycerol in the in presence of 50 uM acetyl Co-A. Acetylated recombinant Foxo1 was bound to GSH beads and beads were washed 2 times in acetylation buffer and 2 times in deacetylation buffer containing 25 Tris HCl (pH 8.0), 137 mM NaCl, 2.7 mM KCl, 1 mM

MgCl2 and incubated with indicated deacetylases for 1 hr at 30 degrees. NAD+ was added to the SIRT1 reaction as previously described (Brunet et al., 2004). Reactions were ran out on SDS-PAGE gel and blotted with indicated antibodies.

Tissue Isolation and Histology

Experimental mice were cervically dislocated and liver was harvested immediately and either processed for histological analysis (10% formalin) or frozen in liquid nitrogen for molecular studies. These samples were then placed frozen into Nunc tubes and homogenized in lysis buffer (20 mM Tris pH 7.5, 150 mM NaCl, 1 mM EDTA, 1 mM EGTA, 1% Triton X-100, 2.5 mM pyrophosphate, 50 mM NaF, 5 mM β-glycero-phosphate, 50 nM calyculin A, 1 mM Na3VO4, Roche complete protease inhibitors) on ice for 30 s using a tissue homogenizer. For glycogen assay on mouse liver, 10 mg or less of frozen livers of corresponding mice were weighed and homogenized using a 2 ml dounce homogenizer and assessed for glycogen content using Glycogen Assay Kit (Biovision). Samples were done in triplicate and read out in triplicates. Data represents the mean+/−SEM. For histology, mouse livers were harvested and fixed in 10% formalin for 24 hrs and then switched to 70% EtOH. Livers were embedded in paraffin and 5 micron liver sections were obtained and stained for hematoxylin and eosin stain or Periodic acid-Schiff (PAS) stain. Slides were viewed on Zeiss microscope and images were taken using CRI Nuance system.

Mouse Luciferase Imaging

Control scrambled shRNA or HDAC shRNAs were co-injected with pAd-G6Pase-luc reporter, pAd-pRenilla-Luc or pAd-RSV-Bgal in 8 week old male C57BL/6J mice. 4 days later, mice were starved for 18 h and imaged on IVIS Kinetic 200 from Caliper Life Sciences following 300 mg/kg D-luciferin injection and anesthetized using isoflurane. Relative photon counts were normalized comparing control shRNA injected mice to HDAC4/5/7 shRNA injected mice using Living Image 3.2 (as well as to B-gal expression). In vivo imaging experiment was repeated three independent times.

Glucose Tolerance Tests

Glucose tolerance tests (GTTs) were performed on 10-12 week old db/db mice injected with either scrambled or HDAC4/5/7 shRNAs. 7-10 days after adenoviral infection, mice were fasted for 18 h overnight, basal fasted blood glucose was measured and mice were injected with 1 g glucose per kg (except B6 mice used 2 g glucose per kg) and blood glucose readings were taken at indicated time points. Pyruvate tolerance tests (PTTs): 16 week db/db mice were starved 18 h overnight, basal fasting blood glucose was measured and mice were injected with 2 g sodium pyruvate per kg. Blood glucose readings were taken at indicated time points.

### REFERENCES

Berdeaux, R., Goebel, N., Banaszynski, L., Takemori, H., Wandless, T., Shelton, G. D., and Montminy, M. (2007). SIK1 is a class II HDAC kinase that promotes survival of skeletal myocytes. Nat Med 13, 597-603.
Biddinger, S. B., and Kahn, C. R. (2006). From mice to men: insights into the insulin resistance syndromes. Annu Rev Physiol 68, 123-158.
Brent, M. M., Anand, R., and Marmorstein, R. (2008). Structural basis for DNA recognition by FoxO1 and its regulation by posttranslational modification. Structure 16, 1407-1416.
Brunet, A., Sweeney, L. B., Sturgill, Chua, K. F., Greer, P. L., Lin, Y., Tran, H., Ross, S. E., Mostoslavsky, R., Cohen, H. Y., et al. (2004). Stress-dependent regulation of FOXO transcription factors by the SIRT1 deacetylase. Science 303, 2011-2015.
Calnan, D. R., and Brunet, A. (2008). The FoxO code. Oncogene 27, 2276-2288
Canettieri, G., Di Marcotullio, L., Greco, A., Coni, S., Antonucci, L., Infante, P., Pietrosanti, L., De Smaele, E., Ferretti, E., Miele, E., et al. (2010) Histone deacetylase and Cullin3-REN(KCTD11) ubiquitin ligase interplay regulates Hedgehog signalling through Gli acetylation. Nat Cell Biol 12, 132-142.
Canto, C., Gerhart-Hines, Z., Feige, J. N., Lagouge, M., Noriega, L., Milne, J. C., Elliott, P. J., Puigserver, P., and Auwerx, J. (2009). AMPK regulates energy expenditure by modulating NAD+ metabolism and SIRT1 activity. Nature 458, 1056-1060.
Canto, C., and Auwerx, J. (2010) AMP-activated protein kinase and its downstream transcriptional pathways. Cell Mol Life Sci. PMID: 20640476
Creemers, E. E., Sutherland, L. B., McAnally, J., Richardson, J. A., and Olson, E. N. (2006). Myocardin is a direct transcriptional target of Mef2, Tead and Foxo proteins during cardiovascular development. Development 133, 4245-4256.
Dansen, T. B., Kops, G. J., Denis, S., Jelluma, N., Wanders, R. J., Bos, J. L., Burgering, B. M., and Wirtz, K. W. (2004). Regulation of sterol carrier protein gene expression by the forkhead transcription factor FOXO3a. J Lipid Res 45, 81-88.
Dentin, R., Liu, Y., Kao, S. H., Hedrick, S., Vargas, T., Heredia, J., Yates. J., 3rd, and Montminy, M. (2007). Insulin modulates gluconeogenesis by inhibition of the coactivator TORC2. Nature 449, 366-369.
Dequiedt, F., Martin, M., Von Blume, J., Vertommen, D., Lecomte, E., Mari, N., Heinen, M. F., Bachmann, M., Twizere, J. C., Huang, M. C., et al. (2006). New role for hPar-1 kinases EMK and C-TAK1 in regulating localization and activity of class IIa histone deacetylases. Mol Cell Biol 26, 7086-7102.
Djouder, N., Tuerk, R. D., Suter, M., Salvioni, P., Thali, R. F., Scholz, R., Vaahtomeri, K., Auchli, Y., Rechsteiner, H., Brunisholz, R. A., et al. (2010). PKA phosphorylates and inactivates AMPKalpha to promote efficient lipolysis. EMBO J 29, 469-481.
Dong, X., Park, S., Lin, X., Copps, K., Yi, X., and White, M. F. (2006). Irs1 and Irs2 signaling is essential for hepatic glucose homeostasis and systemic growth. J Clin Invest 116, 101-114.
Egan, D. F., Shackelford, D. B., Mihaylova, M. M., Gelino, S. R., Kohnz, R. A., Mair, W., Vasquez, D. S., Joshi, A., Gwinn, D. M., Taylor, R., et al. (2010). Phosphorylation of ULK1 (hATG1) by AMP-Activated Protein Kinase Connects Energy Sensing to Mitophagy. Science 331, 456-461
Fischle, W., Dequiedt, F., Hendzel, M. J., Guenther, M. G., Lazar, M. A., Voelter, W., and Verdin, E. (2002). Enzymatic activity associated with class II HDACs is dependent on a multiprotein complex containing HDAC3 and SMRT/N-CoR. Mol Cell 9, 45-57.
Gregoire, S., Xiao, L., Nie, J., Zhang, X., Xu, M., Li, J., Wong, J., Seto, E., and Yang, X. J. (2007). Histone deacetylase 3 interacts with and deacetylates myocyte enhancer factor 2. Mol Cell Biol 27, 1280-1295.
Guarente, L. (2006). Sirtuins as potential targets for metabolic syndrome. Nature 444, 868-874.
Gwinn, D. M., Shackelford, D. B., Egan, D. F., Mihaylova, M. M., Mery, A., Vasquez, D. S., Turk, B. E., and Shaw, R. J. (2008). AMPK phosphorylation of raptor mediates a metabolic checkpoint. Mol Cell 30, 214-226.
Haberland, M., Montgomery, R. L., and Olson, E. N. (2009). The many roles of histone deacetylases in development and physiology: implications for disease and therapy. Nat Rev Genet 10, 32-42.
Haeusler, R. A., Kaestner, K. H., and Accili, D. (2010). FoxOs function synergistically to promote glucose production. J Biol Chem 285, 35245-35248.
Haigis, M. C., and Sinclair, D. A. (2010). Mammalian sirtuins: biological insights and disease relevance. Annu Rev Pathol 5, 253-295.
Houtkooper, R. H., Canto, C., Wanders, R. J., and Auwerx, J. (2010). The secret life of NAD+: an old metabolite controlling new metabolic signaling pathways. Endocr Rev 31, 194-223.
Hurley, R. L., Barre, L. K., Wood, S. D., Anderson, K. A., Kemp, B. E., Means, A. R., and Witters, L. A. (2006). Regulation of AMP-activated protein kinase by multisite phosphorylation in response to agents that elevate cellular cAMP. J Biol Chem 281, 36662-36672.
Hutton, J. C., and O'Brien, R. M. (2009). Glucose-6-phosphatase catalytic subunit gene family. J Biol Chem 284, 29241-29245.
Imai, T., Chambon, P., and Metzger, D. (2000). Inducible site-specific somatic mutagenesis in mouse hepatocytes. Genesis 26, 147-148.
Kahn, B. B., Alquier, T., Carling. D., and Hardie, D. G. (2005). AMP-activated protein kinase: ancient energy gauge provides clues to modern understanding of metabolism. Cell Metab 1, 15-25.
Koo, S. H., Flechner, L., Qi, L., Zhang, X., Screaton, R. A., Jeffries, S., Hedrick, S., Xu, W., Boussouar, F., Brindle, P., et al. (2005). The CREB coactivator TORC2 is a key regulator of fasting glucose metabolism. Nature 437, 1109-1111.
Lahm, A., Paolini, C., Pallaoro, M., Nardi, M. C., Jones, P., Neddermann, P., Sambucini, S., Bottomley, M. J., Lo Surdo, P., Carfi, A., et al. (2007). Unraveling the hidden catalytic activity of vertebrate class IIa histone deacetylases. Proc Natl Acad Sci USA 104, 17335-17340.
Matsumoto, M., Pocai, A., Rossetti, L., Depinho, R. A., and Accili, D. (2007). Impaired regulation of hepatic glucose production in mice lacking the forkhead transcription factor Foxo1 in liver. Cell Metab 6, 208-216.
Matsuzaki, H., Daitoku, H., Hatta, M., Aoyama, H., Yoshimochi, K., and Fukamizu, A. (2005). Acetylation of Foxo1 alters its DNA-binding ability and sensitivity to phosphorylation. Proc Natl Acad Sci USA 102, 11278-11283.
McGee, S. L., van Denderen, B. J., Howlett, K. F., Mollica, J., Schertzer, J. D., Kemp, B. E., and Hargreaves, M. (2008). AMP-activated protein kinase regulates GLUT4 transcription by phosphorylating histone deacetylase 5. Diabetes 57, 860-867.
Montminy, M., Koo, S. H., and Zhang, X. (2004). The CREB family: key regulators of hepatic metabolism. Ann Endocrinol (Paris) 65, 73-75.
Paik, J. H., Ding, Z., Narurkar, R., Ramkissoon, S., Muller, F., Kamoun, W. S., Chae, S. S., Zheng, H., Ying, H., Mahoney, J., et al. (2009). FoxOs cooperatively regulate diverse pathways governing neural stem cell homeostasis. Cell Stem Cell 5, 540-553.
Peng, W. T., Pan, C. J., Lee, E. J., Westphal, H., and Chou, J. Y. (2009). Generation of mice with a conditional allele for G6pc. Genesis 47, 590-594.
Qiang, L., Banks, A. S., and Accili, D. (2010). Uncoupling of acetylation from phosphorylation regulates FoxO1 function independent of its subcellular localization. J Biol Chem 285, 27396-27401.
Renault, V. M., Rafalski, V. A., Morgan, A. A., Salih, D. A., Brett, J. O., Webb, A. E., Villeda, S. A., Thekkat, P. U., Guillerey, C., Denko, N. C., et al. (2009). FoxO3 regulates neural stem cell homeostasis. Cell Stem Cell 5, 527-539.
Rodgers, J. T., Lerin, C., Haas, W., Gygi, S. P., Spiegelman, B. M., and Puigserver, P. (2005). Nutrient control of glucose homeostasis through a complex of PGC-1alpha and SIRT1. Nature 434, 113-118.
Salganik, S. V., Weinstein, D. A., Shupe, T. D., Salganik, M., Pintilie, D. G., and Petersen, B. E. (2009). A detailed characterization of the adult mouse model of glycogen storage disease Ia. Lab Invest 89, 1032-1042.
Schuetz, A., Min, J., Allali-Hassani, A., Schapira, M., Shuen, M., Loppnau, P., Mazitschek, R., Kwiatkowski, N. P., Lewis, T. A., Maglathin, R. L., et al. (2008). Human HDAC7 harbors a class IIa histone deacetylase-specific zinc binding motif and cryptic deacetylase activity. J Biol Chem 283, 11355-11363.
Screaton, R. A., Conkright, M. D., Katoh, Y., Best, J. L., Canettieri, G., Jeffries, S., Guzman, E., Niessen, S., Yates, J. R., 3rd, Takemori, H., et al. (2004). The CREB coactivator TORC2 functions as a calcium- and cAMP-sensitive coincidence detector. Cell 119, 61-74.
Shackelford, D. B., and Shaw, R. J. (2009). The LKB1-AMPK pathway: metabolism and growth control in tumour suppression. Nat Rev Cancer 9, 563-575.
Shaw, R. J., Lamia, K. A., Vasquez, D., Koo, S. H., Bardeesy, N., Depinho, R. A., Montminy, M., and Cantley, L. C. (2005). The kinase LKB1 mediates glucose homeostasis in liver and therapeutic effects of metformin. Science 310, 1642-1646.
van der Linden, A. M., Nolan, K. M., and Sengupta, P. (2007). KIN-29 SIK regulates chemoreceptor gene expression via an MEF2 transcription factor and a class II HDAC. EMBO J 26, 358-370.
Vander Kooi, B. T., Streeper, R. S., Svitek, C. A., Oeser, J. K., Powell, D. R., and O'Brien, R. M. (2003). The three insulin response sequences in the glucose-6-phosphatase catalytic subunit gene promoter are functionally distinct. J Biol Chem 278, 11782-11793.
Viollet, B., Guigas, B., Leclerc, J., Hebrard, S., Lantier, L., Mounier, R., Andreelli, F., and Foretz, M. (2009). AMPK in the regulation of hepatic energy metabolism: from physiology to therapeutic perspectives. Acta Physiol (Oxf) 196, 81-98.
Wang, Z., Zang, C., Cui, K., Schones, D. E., Barski, A., Peng, W., and Zhao, K. (2009). Genome-wide mapping of HATs and HDACs reveals distinct functions in active and inactive genes. Cell 138, 1019-1031.
Wen, Y. D., Perissi, V., Staszewski, L. M., Yang, W. M., Krones, A., Glass, C. K., Rosenfeld, M. G., and Seto, E. (2000). The histone deacetylase-3 complex contains nuclear receptor corepressors. Proc Natl Acad Sci USA 97, 7202-7207.
Witt, O., Deubzer, H. E., Milde, T., and Oehme, I. (2009). HDAC family: What are the cancer relevant targets? Cancer Lett 277, 8-21.
Chen, D., Bruno, J., Easlon, E., Lin, S. J., Cheng, H. L., Alt, F. W., and Guarente, L. (2008). Tissue-specific regulation of SIRT1 by calorie restriction. Genes Dev 22, 1753-1757.
Hsiao, A., Ideker, T., Olefsky, J. M., Subramaniam, S. VAMPIRE microarray suite: a web-based platform for the interpretation of gene expression data. Nucleic Acids Res. 33, W627-W632 (2005).
McKinsey, T. A., Zhang, C. L., Lu, J., and Olson, E. N. (2000a). Signal-dependent nuclear export of a histone deacetylase regulates muscle differentiation. Nature 408, 106-111.
McKinsey, T. A., Zhang, C. L., and Olson, E. N. (2000b). Activation of the myocyte enhancer factor-2 transcription factor by calcium/calmodulin-dependent protein kinase-stimulated binding of 14-3-3 to histone deacetylase 5. Proc Natl Acad Sci USA 97, 14400-14405.
Motta, M. C., Divecha, N., Lemieux, M., Kamel, C., Chen, D., Gu, W., Bultsma, Y., McBurney, M., and Guarente, L. (2004). Mammalian SIRT1 represses forkhead transcription factors. Cell 116, 551-563.
Purushotham, A., Schug, T. T., Xu, Q., Surapureddi, S., Guo, X., and Li, X. (2009). Hepatocyte-specific deletion of SIRT1 alters fatty acid metabolism and results in hepatic steatosis and inflammation. Cell Metab 9, 327-338.
Rodgers, J. T., and Puigserver, P. (2007). Fasting-dependent glucose and lipid metabolic response through hepatic sirtuin 1. Proc Natl Acad Sci USA 104, 12861-12866.
van der Horst, A., Tertoolen, L. G., de Vries-Smits, L. M., Frye, R. A., Medema, R. H., and Burgering, B. M. (2004). FOXO4 is acetylated upon peroxide stress and deacetylated by the longevity protein hSir2(SIRT1). J Biol Chem 279, 28873-28879.
Vega, R. B., Harrison, B. C., Meadows, E., Roberts, C. R., Papst, P. J., Olson, E. N., and McKinsey, T. A. (2004). Protein kinases C and D mediate agonist-dependent cardiac hypertrophy through nuclear export of histone deacetylase 5. Mol Cell Biol 24, 8374-8385.
Zhao, X., Ito, A., Kane, C. D., Liao, T. S., Bolger, T. A., Lemrow, S. M., Means, A. R., and Yao, T. P. (2001). The modular nature of histone deacetylase HDAC4 confers phosphorylation-dependent intracellular trafficking. J Biol Chem 276, 35042-35048.
Martin, M., et al. Int J Dev Biol 53 (2-3), 291 (2009).
Grozinger, C M and Schreiber, S L. Proc Natl Acad Sci USA 97(14), 7835 (2000).
Chang, S. et al. Mol Cell Biol 24(19), 8467 (2004).

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

1. A method of treating a metabolic disorder, comprising administering to a subject in need thereof an inhibitor of a Class IIa histone deacetylase (HDAC).

2. The method of claim 1, wherein the Class IIa HDAC is selected from the group consisting of: HDAC 4, 5, 7, and 9.

3. The method of claim 1, wherein the Class IIa HDAC inhibitor inhibits the activity of at least one Class IIa HDAC selected from the group consisting of: HDAC 4, 5, 7, and 9.

4. The method of claim 3, wherein the Class IIa HDAC inhibitor inhibits the activity of HDAC 4, 5, 7, and 9.

5. The method of claim 1, wherein the metabolic disorder is selected from the group consisting of: diabetes, insulin resistance, and obesity.

6. The method of claim 5, wherein the diabetes is selected from the group consisting of: type 1 diabetes, type 2 diabetes, and gestational diabetes.

7. The method of claim 1, wherein the Class II a HDAC inhibitor lowers blood glucose levels in the subject.

8. The method of claim 1, wherein the Class IIa HDAC inhibitor inhibits expression of a Class IIa HDAC.

9. The method of claim 1, wherein the Class IIa HDAC inhibitor inhibits nuclear localization of a Class IIa HDAC.

10. The method of claim 1, wherein the Class IIa HDAC inhibitor inhibits dephosphorylation of a Class IIa HDAC.

11. The method of claim 1, wherein the Class IIa HDAC inhibitor inhibits Class IIa HDAC mediated deacetylation of a transcription factor.

12. The method of claim 11, wherein the transcription factor is a FOXO transcription factor.

13. A method of screening for a compound for treating a metabolic disorder, comprising expressing a Class IIa HDAC in an isolated hepatocyte or in the liver of a non-human animal in the absence and presence of a test compound, and evaluating the activity of the Class IIa HDAC in the absence and presence of the test compound.

14. The method of claim 13, wherein the activity of the Class IIa HDAC is selected from the group consisting of: expression, localization, phosphorylation state, and FOXO transcription factor deacetylation.

15. The method of claim 1, wherein the Class IIa inhibitor is MC1568 or a pharmaceutically acceptable salt thereof.

16. The method of claim 15, wherein the Class IIa inhibitor lowers blood glucose levels in the subject.

17. The method of claim 15, wherein the metabolic disorder is diabetes.

18. The method of claim 17, wherein the diabetes is selected from the group consisting of: type 1 diabetes, type 2 diabetes, and gestational diabetes.

19. A method of treating a muscle wasting disease, comprising administering to a subject in need thereof an inhibitor of a Class IIa histone deacetylase (HDAC).

20. The method of claim 19, wherein the Class IIa HDAC is selected from the group consisting of: HDAC 4, 5, 7, and 9.

21. The method of claim 19, wherein the Class IIa HDAC inhibitor inhibits the activity of at least one Class IIa HDAC selected from the group consisting of: HDAC 4, 5, 7, and 9.

22. The method of claim 21, wherein the Class IIa HDAC inhibitor inhibits the activity of HDAC 4, 5, 7, and 9.

23. The method of claim 19, wherein the muscle wasting disease is selected from the group consisting of: cachexia, muscle wasting, age-related sarcopenia, and muscular dystrophy.

24. The method of claim 1, wherein the Class IIa HDAC inhibitor inhibits the interaction of one or more Class IIa HDACs with HDAC 3.

25. The method of claim 24, wherein the Class IIa HDAC inhibitor inhibits the interaction of HDAC 4 and/or 5 with HDAC 3.
